(19) United States
(12) Patent Application Publication
(10) Pub. No.: US 2024/0241059 A1
(43) Pub. Date: Jul. 18, 2024

(54) CARTRIDGE, SYSTEM, AND METHOD FOR MOLECULAR DIAGNOSTIC REACTION TESTING

(71) Applicant: Nicoya Lifesciences Inc., Kitchener (CA)

(72) Inventors: Seray CICEK, Kitchener (CA); Yuxiu GUO, Kitchener (CA); Katariina Hanna Zakaaria Sepp JAENES, Kitchener (CA); Lucas ROBINSON, Kitchener (CA); Afifa SALEEM, Mississauga (CA)

(21) Appl. No.: 18/579,845

(22) PCT Filed: Jul. 29, 2022
(86) PCT No.: PCT/CA2022/051170

§ 371 Date: Jan. 16, 2024

Related U.S. Application Data
(60) Provisional application No. 63/227,740, filed on Jul. 30, 2021.

(51) Int. Cl. G01N 21/77; G01N 33/543; G01N 33/569; G01N 35/00; G01N 35/10

(52) U.S. Cl. CPC G01N 21/77; G01N 33/54387; G01N 33/56983; G01N 35/00029; G01N 35/00732; G01N 35/1095; G01N 2021/7786; G01N 2035/00158

(57) ABSTRACT

There are provided systems and methods for molecular diagnostic reaction testing. The method including: receiving a patient sample in a collection device: providing the sample to a microfluidic cartridge via capillary action: inserting the microfluidic cartridge into a diagnostic device: capturing information associated with the sample: performing thermal lysis and inactivation on the sample in the microfluidic cartridge: performing sample mixing with a freeze-dried master mix by passing the sample through microfluidic channels in the microfluidic cartridge that enables mixing of the samples with the freeze-dried master mix: perform multiplex detection of different targets by passing the mixed sample into detection chambers containing probes: capturing images of the mixed samples in the detection chambers: and outputting the images.

### CROSS-REFERENCE

This application claims the benefit of U.S. Provisional Application No. 63/227.740. filed Jul. 30, 2021, which is herein incorporated by reference in its entirety.

### TECHNICAL FIELD

The following relates generally to analytical instruments and more specifically to a cartridge. system, and method for molecular diagnostic reaction testing.

### BACKGROUND

There is a strong need for distributed rapid diagnostics deployed at point-of-need to screen patients and control outbreaks such as the Covid-19 pandemic. These needs range from the detection of the viral particles for diagnosing a disease to measurements of antibodies in samples to determine antibody mediated protective immune response.

A popular approach for nucleic acid detection uses reverse transcriptase quantitative polymerase chain reaction (RT-qPCR). This approach is generally employed in centralized labs and requires approximately three hours to complete. In general. diagnostic testing employed in laboratory settings requires multiple devices and consumables for RNA extraction that. while making the sensitivity and accuracy of the test high. increases the cost of the testing. While RT-qPCR is generally reliable. it requires shipment of the samples to centralized laboratories, where the storage of the reaction reagents in temperature controlled environments need to be accommodated. This system which relies upon centralized laboratories has high cost of operation. requires trained personnel. intensive hours and can incur delays due to shipping and processing times. This workflow ultimately limits the number of tests which can be performed regionally. There is thus a substantial need for easy-to-use testing technologies that can be deployed at point-of-need settings: such as pharmacies. local clinics. workplaces. and schools.

Traditionally. there are two main categories of point-of-need tests used for viral detection: Antigen tests and molecular tests. (1) Antigen tests rely on physical interaction of the viral capsid with detection probes to generate a read-out. Low sensitivity restricts the use of antigen tests and requires a follow-up molecular test verification. Each test requires an operator resulting in limited throughput (2) Molecular tests rely on amplification of the viral genome particles to enable disease diagnosis. Thus, a small amount of virus present in the sample can be detected. RT-qPCR based molecular tests require extraction of the viral genome from the sample and temperature cycling to amplify the target of interest. Alternatively. isothermal molecular tests such as reverse transcriptase loop-mediated amplification (RT-LAMP) technology offer a solution that can deliver diagnostic results in a shorter period of time. RT-LAMP technology utilizes reagents that can withstand harsh conditions, and does not require intensive sample clean-up or temperature cycling, shortening the time between sample collection to result output.

While some RT-LAMP based technologies are available in the market existing colorimetric molecular kits utilize pH based indicators. Based on our clinical studies we have found that pH based indicators are highly prone to false positive results due to pH changes in oral and nasal floras of patients from person to person.

For the detection of antibodies, two commonly used techniques include lateral flow tests for antibody detection and enzyme linked immunosorbent assays (ELISA). Lateral flow tests utilize paper strips to facilitate liquid draw and capture antibodies on immobilized surfaces coated with antigens. While this test can be performed with minimal training, it does not provide quantitative information. ELISA assays also utilize a similar approach in the liquid phase. By conjugating molecules detector antibodies, the test output can be quantified via colorimetric change in enzymatic reaction or fluorescent molecules. Due to the manual steps involved in an ELISA procedure, currently this is limited to laboratory settings and requires highly trained personnel.

Alternatively, enzymatic and chemical reactions play important roles for detecting chemical concentrations of small molecules to aid medical decision-making. Examples include detection of magnesium ions with eriochrome black T and measuring glucose levels. While some of these are made largely accessible with innovations such as glucose-meter, the majority still require a laboratory setting for analysis.

### SUMMARY

In various aspects, there is a system and method provided for molecular diagnostic reaction testing that can specifically detect different targets, including nucleic acid and proteins, from a patient sample and minimize user errors such as contamination. The method comprising: receiving a patient sample in a collection device: providing the sample to a microfluidic cartridge via capillary action: inserting the microfluidic cartridge into a diagnostic device capable of receiving multiple cartridges: capturing information associated with the sample cup and test cartridges via a computer-readable code (including QR code or barcode): performing sample extraction and inactivation on the sample in the microfluidic cartridge via thermal and chemical lysis: performing sample mixing with a freeze-dried master mix by passing the sample through microfluidic channels in the microfluidic cartridge that enables mixing of the samples with the freeze-dried master mix: performing multiplex detection of different targets by passing the mixed sample into different detection chambers containing probes: incubating the cartridge for a previously defined time at a defined temperature: capturing images of the detection chambers: outputting images and related quantitative data information; and uploading the image and data result to the cloud database for cloud computing and storage.

Patient sample collection can alternatively be assisted by cartridge and diluted by a buffer loaded in the sample cup.

Patient sample collection can include swabbing of the surfaces including nasal or oral, collection of bodily fluids such as blood, urine, sweat or assisted by a diluent such as gargling of saline or nasal wash.

Probes used to detect targets of interest can include primers and/or capture antibodies that can be stored in liquid format or immobilized or solidified to different matrices such as cartridge surface or a porous matrix such as paper or gel.

Freeze dried master mix can include antibodies, enzymes, small molecules (deoxyribonucleotide triphosphate), buffering agent or gold nanoparticles required to facilitate the signal change in image output.

Sample cup can be linked to a patient reference number and operator by registering the computer readable unique identifier such as QR code to an online portal to facilitate quality traced sample collection.

Cartridge information can be linked to a protocol and quality information, such as serial number and batch number, in order to automate processing of samples and match test results with patient reference number pseudonymously.

Cartridge can contain two wire leads capable of receiving voltage differential to detect the presence of the cartridge by an external device and move charged particles inside the device reversibly.

In one aspect, the presently disclosed invention is directed to a method for molecular diagnostics, the method comprising: receiving a patient sample in a collection device: coupling the collection device to a microfluidic cartridge: dispensing the patient sample into the microfluidic cartridge using capillary action: inserting the microfluidic cartridge into a diagnostic system: detecting the microfluidic cartridge and initiating a sample testing protocol, wherein initiating the sample testing protocol comprises: reading a computer readable code located on the microfluidic cartridge: receiving a sample test protocol from a computer system based on the computer readable code: and performing the sample test protocol: identifying a detection chamber or imaging chamber on the microfluidics device: capturing image data of the patient sample in the detection chamber or imaging chamber at one or more time points during an incubation period based on the sample test protocol: performing image analysis on the captured image data: and outputting a diagnostic result based on the image analysis.

In some embodiments, the microfluidic cartridge comprises a sensor having a sensor surface, and optionally wherein the sensor surface comprises a cartridge surface, an immobilization surface or a porous paper matrix. In one, the sensor surface comprises an immobilization surface, and wherein the immobilization surface contains agarose, gelatin, alginate, optical fiber, plastic surface or paper matrices.

In some embodiments, the detection chamber or imaging chamber comprises one or more regions of interest.

In some embodiments, the microfluidic cartridge contains pillars, a porous matrix or membrane for separation by size exclusion of particles larger than viral particles.

In some embodiments, the sample testing protocol comprises one or more test parameters selected from assay conditions, assay temperature, incubation time, image capture parameters, illumination sources, optical filters or any combination thereof. In one embodiment, the image capture parameters comprise fluorescent, luminescent or colorimetric, and wherein the captured image data is fluorescent data, colorimetric data, wavelength data, bioluminescent data or chemiluminescent data.

In some embodiments, the sample testing protocol comprises the use of one or more reagents and wherein the one or more reagents are added to the patient sample to create a reaction sample. In one embodiment, the one or more reagents comprises probes or primers, and wherein the probes or primers are stored in a liquid medium or immobilized to the sensor surface. In another embodiment, the one or more reagents comprises capture antibodies, and wherein the capture antibodies are stored in a liquid medium or immobilized to the sensor surface. In still another embodiment, the one or more reagents comprises a freeze-dried master mix comprising antibodies, enzymes or gold nanoparticles, and wherein the antibodies, enzymes or gold nanoparticles facilitate a change in the captured image data. In still yet another embodiment, the freeze-dried master mix is mixed with the patient sample by passing the patient sample through a microfluidic channel in the microfluidic cartridge containing the freeze-dried master mix thereby creating a reaction sample by mixing of the patient sample with the freeze-dried master mix. In still another embodiment, the one or more reagents comprises a colorimetric reagent and/or a hydrogen peroxide reagent, and wherein the colorimetric reagent and/or hydrogen peroxide reagent are stored on the microfluidic cartridge in one or more reagent storage compartments.

In some embodiments, the testing protocol further comprises inactivating the sample in the microfluidic cartridge, and wherein the inactivation comprises chemical, physical or thermal inactivation. In one embodiment, chemical inactivation comprises incubation with a detergent and/or chelating agent. In another embodiment, physical inactivation comprises sonication.

In some embodiments, the testing protocol further comprises performing multiplexed detection of different targets by passing the sample into different detection chambers, optionally wherein each of said detection chambers comprises a different reagent.

In some embodiments, the computer system comprises a processor, storage and computer readable code, and wherein the computer readable code includes one or more sample testing protocols.

In some embodiments, the sample collection device further comprises a computer readable code, and wherein the sample collection device computer readable code is read and linked to a patient sample or patient reference number to facilitate tracking of the patient sample and wherein optionally the computer readable code is used as a token to communicate with an external database. In one embodiment, the microfluidic cartridge computer readable code is further linked to test quality information including but not limited to a cartridge serial number to facilitate automate processing of the patient sample.

In some embodiments, the diagnostic results are stored in the cloud, and optionally wherein the diagnostic results further comprise an associated reference number, one or more quality information features, such as batch number, expiry date, lot number, or successful analysis threshold, and one or more operator information features, such as operator ID, and optionally wherein the test results are matched with an external database for patient identification.

In some embodiments, the microfluidic cartridge comprises two wire leads capable of receiving a voltage differential in order to move charged particles inside the device reversibly. In one embodiment, the microfluidic cartridge comprises two wire leads capable of receiving a voltage differential in order to heat, lyse particles, or for flow control by manipulating temperature or voltage sensitive materials.

In some embodiments, the diagnostic device contains electrical connectors that mate with leads of an external device to detect the presence of the microfluidic cartridge.

In another aspect, the presently disclosed invention is directed to a system for molecular diagnostics, the system comprising: providing a microfluidic cartridge loaded with a patient sample, wherein the microfluidic cartridge includes a computer readable code, and a detection chamber or imaging chamber: a means for identifying and reading a computer readable code located on the microfluidic cartridge: a computer system comprising a processor, storage and computer readable code, wherein the computer readable code includes one or more sample testing protocols: and an image module comprising an imaging capture device for capturing one or more images from the detection chamber or imaging chamber of the microfluidic cartridge: and wherein one of the sample testing protocols is selected based on the computer readable code, and wherein the selected sample testing protocol is initiated by the processor based on instructions contained within the computer readable code.

In some embodiments, the microfluidic cartridge comprises a sensor having a sensor surface, and optionally wherein the sensor surface comprises a cartridge surface, an immobilization surface or a porous paper matrix. In one embodiment, the sensor surface comprises an immobilization surface, and wherein the immobilization surface contains agarose, gelatin, alginate, optical fiber, plastic surface or paper matrices.

In some embodiments, the detection chamber or imaging chamber comprises one or more regions of interest.

In some embodiments, the microfluidic cartridge contains pillars, a porous matrix or membrane for separation by size exclusion of particles larger than viral particles.

In some embodiments, the sample testing protocol comprises one or more test parameters selected from assay conditions, assay temperature, incubation time, image capture parameters, illumination sources, optical filters or any combination thereof. In one embodiment, the image capture parameters comprise fluorescent, luminescent or colorimetric, and wherein the captured image data is fluorescent data, colorimetric data, wavelength data, bioluminescent data or chemiluminescent data.

In some embodiments, the sample testing protocol comprises the use of one or more reagents and wherein the one or more reagents are added to the patient sample to create a reaction sample. In one embodiment, the one or more reagents comprises probes or primers, and wherein the probes or primers are stored in a liquid medium or immobilized to the sensor surface. In another embodiment, the one or more reagents comprises capture antibodies, and wherein the capture antibodies are stored in a liquid medium or immobilized to the sensor surface. In another embodiment, the one or more reagents comprises a freeze-dried master mix comprising antibodies, enzymes or gold nanoparticles, and wherein the antibodies, enzymes or gold nanoparticles facilitate a change in the captured image data. In still another embodiment, the freeze-dried master mix is mixed with the patient sample by passing the patient sample through a microfluidic channel in the microfluidic cartridge containing the freeze-dried master mix thereby creating a reaction sample by mixing of the patient sample with the freeze-dried master mix. In still yet another embodiment, the one or more reagents comprises a colorimetric reagent and/or a hydrogen peroxide reagent, and wherein the colorimetric reagent and/or hydrogen peroxide reagent are stored on the microfluidic cartridge in one or more reagent storage compartments.

In some embodiments, the testing protocol further comprises inactivating the sample in the microfluidic cartridge, and wherein the inactivation comprises chemical, physical or thermal inactivation. In one embodiment, chemical inactivation comprises incubation with a detergent and/or chelating agent. In another embodiment, physical inactivation comprises sonication.

In some embodiments, the testing protocol further comprises performing multiplexed detection of different targets by passing the sample into different detection chambers, optionally wherein each of said detection chambers comprises a different reagent.

In some embodiments, the system further comprises a sample collection device, and wherein the sample collection device further comprises a computer readable code, and wherein the sample collection device computer readable code is read and linked to a patient sample or patient reference number to facilitate tracking of the patient sample and wherein optionally the computer readable code is used as a token to communicate with an external database. In one embodiment, the microfluidic cartridge computer readable code is further linked to test quality information including but not limited to a cartridge serial number to facilitate automate processing of the patient sample.

In some embodiments, the imaging system is used to capture image data of the patient sample in the detection chamber or imaging chamber at one or more time points during an incubation period based on the sample test protocol: the computer system is used to perform image analysis on the captured image data: and outputting a diagnostic result based on the image analysis.

In some embodiments, the diagnostic results are stored in the cloud, and optionally wherein the diagnostic results further comprise an associated reference number, one or more quality information features, such as batch number, expiry date, lot number, or successful analysis threshold, and one or more operator information features, such as operator ID, and optionally wherein the test results are matched with an external database for patient identification.

In some embodiments, the microfluidic cartridge comprises two wire leads capable of receiving a voltage differential in order to move charged particles inside the device reversibly. In one embodiment, the microfluidic cartridge comprises two wire leads capable of receiving a voltage differential in order to heat, lyse particles, or for flow control by manipulating temperature or voltage sensitive materials.

In some embodiments, the diagnostic device contains electrical connectors that mate with leads of an external device to detect the presence of the microfluidic cartridge.

In another aspect, the presently disclosed invention is directed to a method for collecting a sample and transferring the sample to a microfluidic cartridge, wherein the method comprises: providing a sample collection funnel, a sample collection cup and a microfluidic cartridge: attaching the sample collection funnel to the sample collection cup: dispensing a patient sample comprising a bodily fluid into the funnel: removing the funnel from the sample collection cup: attaching the sample collection cup to the microfluidic cartridge: and dispensing the patient sample into the microfluidic cartridge using capillary action.

In one embodiment, the bodily fluid is gargle, mouth rinse, sweat, blood or urine. In another embodiment, the sample collection funnel is attached to the sample collection cup using a threaded mating or press fit. In still another embodiment, the sample collection cup is attached to the microfluidic device using a threaded mating or press fit.

In another aspect, the presently disclosed invention is directed to a method for collecting a sample and transferring the sample to a microfluidic cartridge, wherein the method comprises: providing a sample collection device comprising a reagent chamber and a sample collection cup: filling the reagent chamber of the sample collection device with a sample collection medium: inserting the sample collection device into a nasal opening of a test subject: dispensing the sample collection medium, from the reagent chamber, into the nasal opening: collecting in the sample collection cup a patient sample by collecting any fluid that passes from the nasal opening after the sample collection medium is dispensed into the nasal opening: attaching the sample collection cup to a microfluidic cartridge: and dispensing the patient sample into the microfluidic cartridge using capillary action. In one embodiment, the sample collection device further comprises a sample collection funnel to collect the patient sample fluid.

In another aspect, the presently disclosed invention is directed to a method for collecting a blood sample from a patient, wherein the method comprises: providing a sample collection device comprising a collection cartridge and a sample cup, wherein the collection device comprises one or more capillary channels for collecting a blood sample from a patient, and wherein the sample collection cup comprises a sample collection reagent: pricking a finger of a patient thereby drawing blood, and bringing the blood from finger prick in contact with an edge of the collection cartridge: collecting blood in the collection cartridge using capillary action: inserting the collection cartridge into the sample cup containing the sample collection reagent: drawing the sample collection reagent from the sample cup into the capillary channels of the sample cartridge by using capillary action.

These and other aspects are contemplated and described herein. It will be appreciated that the foregoing summary sets out representative aspects of embodiments to assist skilled readers in understanding the following detailed description.

### DESCRIPTION OF THE DRAWINGS

The features of the invention will become more apparent in the following detailed description in which reference is made to the appended drawings wherein:

FIG. 1 is a diagram of a system for molecular diagnostic reaction testing, according to an embodiment:

FIG. 2 is a flowchart for molecular diagnostic reaction testing, according to an embodiment:

FIG. 3 is a chart illustrating correlation between RT-LAMP using a freeze-dried formulation with RT-qPCR for the detection of SARS-COV-2 from patient samples (positive and negative):

FIG. 4 illustrates a sensitivity test of RT-LAMP using the freeze-dried formulation;

FIG. 5 illustrates performance of RT-LAMP using the freeze-dried formulation for the detection of SARS-COV-2 from gargle and swish patient samples:

FIG. 6 illustrates performance of RT-LAMP using the freeze-dried formulation for the detection of SARS-COV-2 from gargle and swish patient samples:

FIG. 7 is a chart showing freeze dry on LAMP reagents:

FIG. 8 is a chart showing amplification of RNA using primers immobilized in gelatin;

FIG. 9 is a chart showing RT-LAMP Agarose Spike-In Tests:

FIG. 10 is a chart showing RT-LAMP Agarose Spike-In Inhibition Tests:

FIG. 11 is a chart showing RT-LAMP Agarose Testing that is normalized;

FIG. 12 is a chart showing RT-LAMP Fluorescence Solid Agarose Tests:

FIG. 13 a chart showing Freeze Dried Fluorescence Primer-Solid Dye Sensitivity Tests:

FIG. 14 illustrates results for a protocol for agarose formulation;

FIG. 15 illustrates a schematic design for a microfluidic cartridge:

FIG. 16 illustrates a perspective view of the microfluidic cartridge:

FIG. 17 illustrates an example of capillary-driven flow through upper subcomponent of microfluidic device:

FIG. 18 illustrates an example of capillary-driven flow through master mix bead mixing chamber:

FIG. 19 illustrates an example of capillary-driven flow through sequential trifurcation over time:

FIG. 20 illustrates an example of serpentine imaging chamber over time:

FIG. 21 illustrates a flow diagram of an example master fabrication process:

FIG. 22 illustrates a flow chart of an example device fabrication and assembly process:

FIG. 23 illustrates a diagram of a mouth rinse collection device: and

FIG. 24 illustrates a diagram of a nasal rinse collection device

FIG. 25 illustrates an assembly method of the sample cup and cartridge

FIG. 26 illustrates a method of blood collection and assembly with a cup containing sample diluent

FIG. 27 illustrates data collected using primer immobilized onto paper surfaces

FIG. 28 illustrates an antibody detection embodiment of the cartridge

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”: singular articles and pronouns as used throughout include their plural forms, and vice versa: similarly, gendered pronouns include their counterpart pronouns so that pronouns should not be understood as limiting anything described herein to use, implementation, performance, etc. by a single gender: “exemplary” should be understood as “illustrative” or “exemplifying” and not necessarily as “preferred” over other embodiments. Further definitions for terms may be set out herein: these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM. EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

Point-of-need diagnostic testing devices for viral detection can be generally categorized into two types: antigen tests and molecular tests.

Antigen tests generally rely on physical interaction of a viral capsid with detection probes to generate a read-out. Due to the one-on-one interaction of an analyte with a virus, these tests suffer from sensitivity issues. Low sensitivity restricts the use of antigen tests to scenarios where the lack of sensitivity is compensated by frequency and verified by a molecular test as a follow-up. In this way, use of antigen tests for travel screening is generally not recommended due to the increased risk associated with missing a positive case. In addition, antigen tests are generally only approved to work with nasal/nasopharyngeal samples that often require a trained nurse or physician to collect the sample. Increased frequency requirement and healthcare professionals requirement for sample collection result in significant costs.

Molecular tests generally rely on amplification of the viral genome particles to enable disease diagnosis. Due to amplification, single digit (3-9) copies present in an analyzed sample can be detected. RT-qPCR based molecular tests require extraction of a viral genome from the sample and temperature cycling to amplify the target of interest. This often results in slower results (e.g., just under an hour) compared to antigen based rapid tests (e.g., 20 minutes). Additionally, RT-qPCR tests generally require significant capital cost.

Isothermal molecular tests, such as reverse transcriptase loop-mediated amplification (RT-LAMP) technology, provide a molecular test that can deliver diagnostic results substantially quicker (e.g., around 45 minutes). RT-LAMP technology utilizes reagents that can withstand harsh conditions, and does not require intensive sample clean-up or temperature cycling. Some RT-LAMP based technologies utilize pH based indicators. However, pH based indicators are highly prone to false positive results due to pH changes in oral and nasal fluids of patients.

In addition, viral RNA extraction kits (such as silica column-based extraction kits or magnetic bead-based extraction kits) can be used to obtain RNA from saliva and nasopharyngeal samples. However, these kits are generally expensive, generally require additional expensive and bulky lab equipment, are generally time-intensive, and generally require trained personnel to operate. Alternatively, samples could be added directly to molecular reactions without purification, however that results in carryover material from the sample matrix, which is highly variable from sample to sample and can affect test results. RNA obtained from extraction kits can be manually added to pH-based indicator dyes: however, such dyes generally have draw backs, as both nasopharyngeal and saliva samples have variable pHs that can affect the dye, resulting in false positives and false negatives. This is particularly an issue when using samples directly without any upstream purification.

For the detection of antibodies, two commonly used techniques include lateral flow tests for antibody detection and enzyme linked immunosorbent assays (ELISA). Lateral flow tests utilize paper strips to facilitate liquid draw and capture antibodies on immobilized surfaces coated with antigens. While this test can be performed with minimal training, it does not provide quantitative information. ELISA assays also utilize a similar approach in the liquid phase. By conjugating molecules detector antibodies, the test output can be quantified via colorimetric change associated with an enzymatic reaction or fluorescent molecules. Due to the manual steps involved in an ELISA procedure, currently this is limited to laboratory settings and requires highly trained personnel.

Alternatively, enzymatic and chemical reactions play important roles for detecting chemical concentrations of small molecules to aid medical decision-making. Examples include detection of magnesium ions with eriochrome black T and measuring glucose levels. While some of these are made largely accessible with innovations such as glucose meter, the majority of reactions still require a laboratory setting for analysis.

The present embodiments provide an RT-LAMP based molecular test that can be deployed at point-of-need and can conduct virus detection (e.g., SARS-COV-2) in oral samples (such as with mouth rinse). Advantageously, the present embodiment illustrates antibody detection using ELISA assay by modifying reagents. Advantageously, the present embodiments allow users to easily collect samples without generally requiring close contact with individuals suspected of being infected with a transmissible virus. Also advantageously, the present embodiments can determine positive test results substantially faster (e.g., less than 30 minutes) over other RT-LAMP reaction approaches (e.g., around 45 minutes). The present embodiments facilitate seamless testing and data management at a point-of-need, remote from a testing facility, and can be performed by any trained users, instead of healthcare professionals.

The present embodiments leverage RT-LAMP for point of care diagnostics through sample preparation. Approaches using RNA purification generally rely upon a centralized lab, and therefore, the present embodiments use patient samples directly. The samples can be collected in various media (phosphate-buffered saline (PBS), viral transport media (VTM), saline, etc.).

In some cases, a pre-treatment can be used in order to maximize sensitivity. Pre-treatments can include, for example, heat treatment, reducing agents (TCEP/DTT), proteinase K, detergents or chelating agents.

Colorimetric RT-LAMP generally requires an indicator for DNA amplification. A particular approach is to measure pH changes in the reaction mixture. For example, phenol-red dye turns from red to yellow upon DNA amplification due to the drop in pH. However, for direct patient samples, there is generally inherent pH variance and therefore is not a useful approach. Colorimetric RT-LAMP can also use indicators which change color upon changing concentration of divalent metallic cations (e.g., Mg++). Examples of this are Hydroxynapthol blue or Eriochrome Black T.

A notable issue with present approaches for RT-LAMP is a high false positivity rate. There have been different approaches to combat this issue: for example, using sequence-specific indicators or molecular fluorescent probes. These options are less prone to false positives but are significantly more expensive.

Embodiments of the present disclosure overcome the challenges in the prior art by using computer vision in a point-of-need diagnostic device using thermal insulations, microfluidic based zero liquid handling and enhanced system interfaces. Various embodiments include the ability to separate 95-degree thermal lysis from 65-degree reaction chamber via introducing a sliding metallic holder. Additionally, various embodiments use a microfluidic device that automates sample concentration, lysis, and mixing of the freeze-dried reagents. The room temperature stable freeze-dried reagents are mixed with solid-phase reagents to enable temperature-dependent reaction. Examples of the solidifying medium are agarose gel or BSA treated Whatman filter paper. In various embodiments, the system interface can be used to seamlessly match sample ID with cartridge information and test results: and in some cases, integrate with electronic medical records. In addition, the system interface can use a camera to recognize a computer-readable code (e.g., QR code) on a sample cartridge to log user and protocol information associated with the molecular test; and can be used to recognize region of interests (ROI) automatically (see, e.g., WO 2021/168578, which was filed Feb. 26, 2021 and is hereby incorporated by reference).

Embodiment of the present disclosure utilizes the computer-readable code (e.g., QR code) on cartridge defines the test type, quality information and protocols to be performed in the receiving device. By uploading the cartridge information to the cloud, protocol steps for the device operation will be returned to the device including determining the reaction parameters (i.e. fluorescent, luminescent or colorimetric) and capturing image data from one or more regions of interest on the cartridge. In one embodiment, the system or device then sends raw image data to the cloud for data analysis, followed by the final qualitative and quantitative result returned back to the device for displaying. Alternatively, data analysis can occur within the device or system itself.

Embodiment of the present disclosure utilizes the computer-readable code (e.g., QR code) on a sample collection cup that uniquely identifies the sample cup. By scanning the computer-readable code, the sample cup number can be linked to additional external information including but not limited to: a reference number that identifies pseudonymized patient information in an external database and operator information.

Embodiments of the present disclosure can use various suitable sample collection approaches using saline, such as: (1) oral collection assistant, and (2) nasal wash. A sample collection kit can include a sealed-off saline tube, a cryovial, a funnel with threaded attachment to cryovial and/or test cartridge. While the present disclosure generally provides liquid based sample collection, it is understood that the present embodiments can be used with any suitable sample collection approach, including nasopharyngeal swabs. In an example for the present embodiments, a test kit for detection of COVID-19 can include a tube or plate with freeze-dried reagents that can detect COVID-19 with the SARS-COV-2 genes such as ORF1A and E1 genes, and the internal control such as human actin gene. A cartridge for detection of COVID-19 can include a cartridge with freeze-dried reagents that can detect COVID-19 with the SARS-COV-2 ORF1A and E1 genes and human actin gene as the internal control. The collected sample can generally be any bodily fluid collected, for example, from the mouth, nose, blood stream, etc. For example, the bodily fluid can be gargle, mouth rinse, saliva, sweat, nasal mucus, blood or urine.

Advantageously, the cartridge can be utilized to facilitate blood collection via capillary action. This can be achieved by gliding a pricked finger over the cartridge edge to collect sample via capillary action and can be followed by coupling the cartridge and the sample cup to introduce dilution buffers to the system.

Advantageously, the present embodiments provide a combination of sample extraction and molecular diagnostic reaction using a single cartridge that is relatively easy to operate, and provides a coupled identification to automate test result delivery. In this way, the complexity of performing the diagnostic tests, and the overall cost and time to obtain test results, are significantly reduced. Also advantageously, the present embodiments only require the cartridge and the diagnostic device, such that there is no need for additional lab equipment (centrifuge, hot plate, pipettes, etc.) and trained technicians: further reducing the cost of performing these diagnostic tests and enabling point-of-care use. Also advantageously, the present embodiments use an hydrogel formulation (such as agarose) of a non-pH dye for use in a molecular reaction that is not sensitive to the pH of the reaction: which can enable direct to sample testing using RT-LAMP. Also advantageously, the present embodiments use a significantly more comfortable approach to collecting samples via mouth gargle or nasal saline wash.

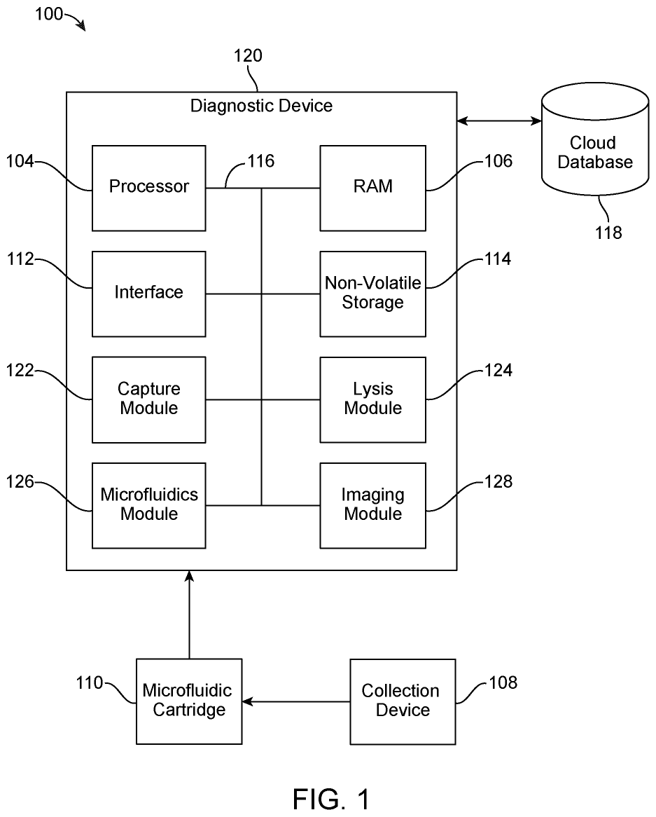

Referring now to FIG. 1, a system for molecular diagnostic reaction testing 100, in

accordance with an embodiment, is shown, FIG. 1 shows various physical and logical components of an embodiment of the system 100. As shown, the system 100 includes a diagnostic device 102 that has a number of physical and logical components, including a processor 104 (comprising one or more processors), random access memory (“RAM”) 106, an interface 112, non-volatile storage 114, and a local bus 116 enabling the processor 104 to communicate with the other components. In some cases, at least some of the one or more processors can be a microprocessor, a system on chip (SoC), a single-board computer (e.g., a Raspberry Pi™), or the like. RAM 106 provides relatively responsive volatile storage to the processor 104. The interface 108 enables interaction with the diagnostic device via input devices or via communication links with other devices: such as other computing devices and servers remotely located from the system 100, such as for a cloud-computing storage. Non-volatile storage 114 stores the operating system and modules, including computer-executable instructions for implementing the operating system and modules, as well as any data used by these services. Additional stored data can be stored in a database 118, which may local or remote (e.g., in the cloud). The diagnostic device 120 further includes a number of physical or conceptual modules that can be executed on their own or on the processor 104; in some embodiments, a capture module 122, a lysis module 124, a microfluidics module 126, and an imaging module 128. The system further includes a microfluidic cartridge 110 and a collection device 108 to be provided to the diagnostic device 120.

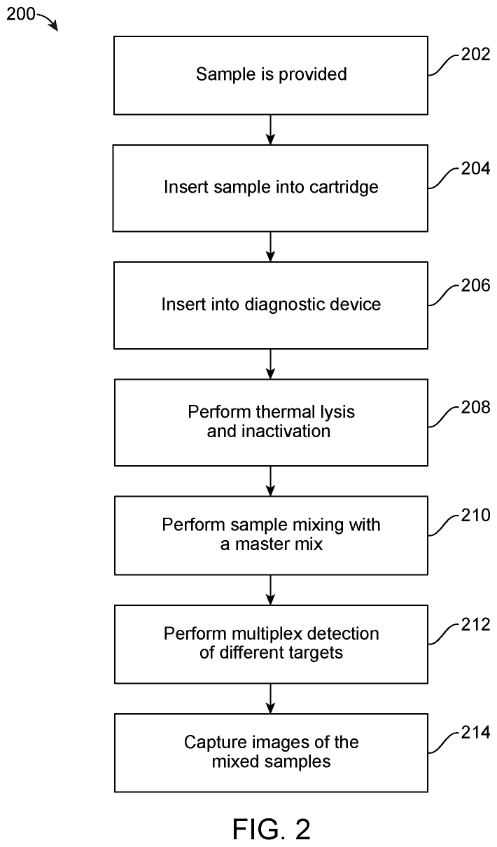

Turning to FIG. 2, a flowchart of a method for molecular diagnostic reaction testing 200, in accordance with an embodiment, is shown.

At block 202, a patient provides a sample in a collection device 108 which is provided to a microfluidic cartridge 110. For example, the patient is given 5 ml of saline and performs a mouth rinse. The mouth rinse can include any suitable approach, for example, three rounds of a 5-second swish followed by a 5-second gargle. After completion, mouthwash is transferred into a collection device 108. In other cases, patient samples can be collected through a syringe-based sinus rinse collector, which collects high viral load samples by repeat nasal rinse, as described herein. The collection device 108 containing the sample is delivered into the microfluidic cartridge 110. In some cases, the outer surface is cleaned to prevent contamination. Advantageously, this sample collection can be performed away from other individuals to avoid communicating transmissible material to them. The cartridge can then be provided to the relevant healthcare staff.

At block 204, the microfluidic cartridge 110 containing the sample is inserted into the diagnostic device 120.

At block 206, the capture module 122 of the diagnostic device 120 performs data capture (e.g., image capture) to determine information about the sample. The capture module 122 comprises one or more input devices, such as a camera or a RFID reader. In a particular case, the first computer readable code (such as a barcode) is located on the collection device 108. This first computer readable code can be linked to an identification of the patient. The first computer readable code can be matched with a second computer readable code (such as a QR code) that resides on the cartridge 110. The second computer readable code associated with the cartridge 110 allows the capture module 122 to receive information from the input device to recognize the first computer readable code and/or the second computer readable code to determine testing and analysis protocol facilitating automation. This can be used to anonymize patient information and allow seamless set-up of test protocol for the device.

At block 208, upon insertion of the test cartridge, the lysis module 124 of the diagnostic device 120 performs thermal lysis and inactivation of the viral particles. The lysis is enabled by heat treatment, for example, 95-degree treatment. This heat lysis can be enabled with the addition of TCEP (tris(2-carboxyethyl)phosphine) and EDTA (ethylenediaminetetraacetic acid) to reduce potential RNase activity. Electrical concentration application can be used to allow a viral genome to be separated from bulky contaminants. Within a certain time period, the viral genome extraction is accomplished.

At block 210, the microfluidics module 126 of the diagnostic device 120 performs sample mixing with a freeze-dried master mix. The sample is passed through microfluidic channels in the diagnostic device 120 via capillary action that enables mixing of the samples with the master mix. The sample then mixes with a reagents mix containing probes and dye that is pre-solidified.

At block 212, the microfluidics module 126 of the diagnostic device 120 performs multiplex detection of different targets. Facilitated by the capillary flow, the samples, mixed with the master mix, are passed into detection chambers, in the diagnostic device 120, that contain solidified probes and dye. The solidified probes are capable of detecting one or multiple target RNA/DNA sequences of interest.

At block 214, the imaging module 128 of the diagnostic device 120 captures images of the mixed samples using an image capturing device (such as a camera). In an example, an image is captured every minute up to 45 minutes. The captured images can then be outputted, such as communicated over a network to a cloud storage. The captured images can also be analyzed, either on the diagnostic device 120 or on another computing system (such as a computing system in communication with the cloud storage). The analyzed data can be stored on an anonymous database that associates the second machine readable code and/or the first machine readable code for privacy reasons. When matched with a patient identification database, test results for the patient can be determined. After imaging, the microfluidic cartridge 110 can be disposed of,

In some embodiments, the diagnostic device 120 can receive multiple microfluidic cartridges 110 at once to process multiple samples simultaneously.

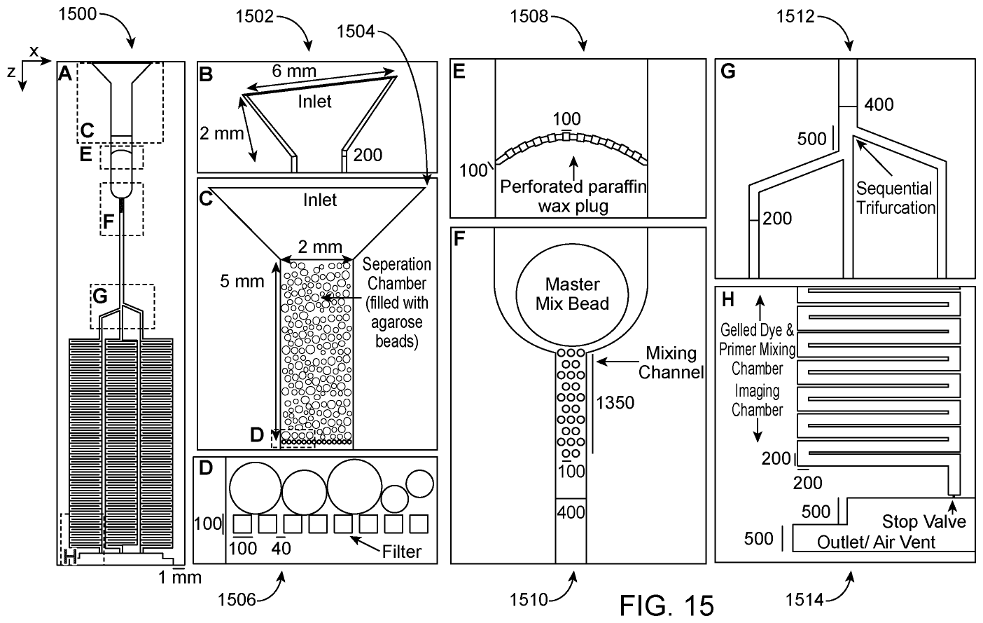

FIG. 15 illustrates an example embodiment of microfluidic cartridge 110; example dimensions are in μm, unless otherwise noted. Diagram 1500 illustrates a front view of the diagnostic device 120, which measures within 12×48 mm×3 mm. Diagram 1502 illustrates a cone-shaped inlet coupled with a wide air vent (in diagram 1510) that induces capillary-driven fluid flow within the device. Diagram 1504 illustrates a separation chamber with a sample separating agent, such as 1% agarose beads that are 45-160 μm in diameter. Diagram 1506 illustrates a built-in filter with 100×100 μm pillars that prevent beads from advancing in the device. Diagram 1508 illustrates perforated paraffin wax plug delays fluid flow until it has cooled. Diagram 1510 illustrates a nozzle-style channel holding a master mix bead that will dissolve in the fluid as it moves through into a narrower channel (for example, 400 μm wide). Mixing of the fluid occurs by movement through pillars of 100 μm diameter. Diagram 1512 illustrates a sample fluid that is separated into narrower channels (for example, 200 μm wide) through a sequential trifurcation. Diagram 1514 illustrates solidified primer and dye in an imaging chamber over a length of 20 mm, where the sample is imaged. A stop valve at the end of the channel and abrupt geometry changes prevent fluid leakage and backflow.

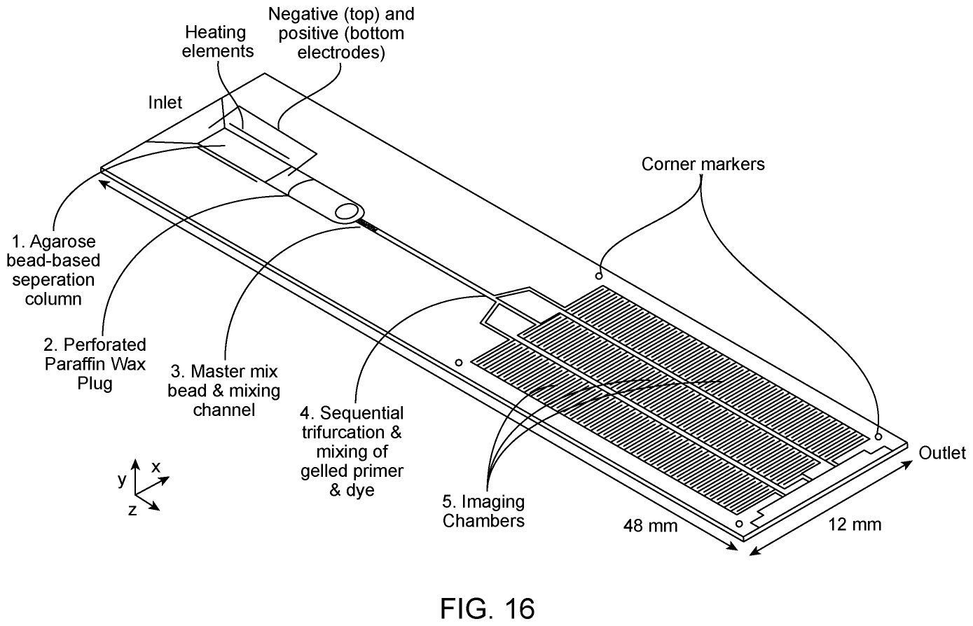

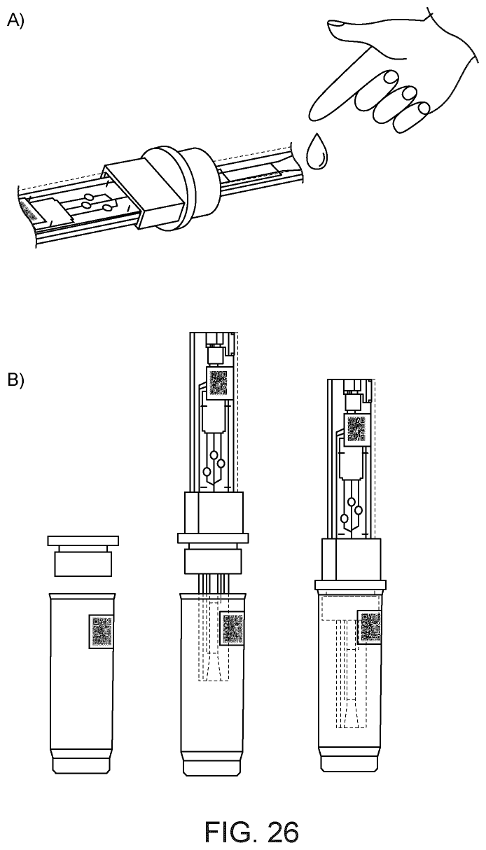

FIG. 16 illustrates a perspective diagram of the example embodiment of the microfluidic cartridge 110 with integrated hardware components. In this example, the device measures 12×48×0.2 mm. The microfluidic cartridge 110 includes a vertical flow network with a 6×0.2 mm inlet and 10×0.2 mm outlet, enabling sample fluid to travel down the channels passively via capillary-driven flow. The cartridge is made of five primary components, connected in series, through which the sample is processed and images are retrieved. These components are as follows:
DNA separation column (shown in diagram 1504)—The fluid first flows into a 2×5×0.2 mm column with a separation agent, such as 1% agarose beads, which have a nucleic acid exclusion limit of 3 kilo base pairs (kbp). Agarose beads can be used as the sieving matrix due to their large surface area, dimensional flexibility, and ease of loading required for a point-of-care setting. The agarose beads have a diameter of 50-150 μm and are contained within the column by a horizontal filter. The filter is made of 14 rectangular columns (100×100×200 μm), separated by 15 40 μm-wide channels allowing only filtered fluid to pass through. Alternative sieving matrices - such as pillar array columns prepared as part if the microfluidic channels or a glass fiber filter paper placed orthogonal to fluid flow can also be used. Through either sieving matrix, viral lysis and electrophoresis occur with the addition of a heating component and 2 electrodes positioned at the top and bottom of the separation column.
Perforated paraffin wax plug (shown in diagram 1508)—The paraffin wax will temporarily prevent flow in the channel while the sample fluid cools below 65° C. As the heated fluid sits above the wax, it will gradually melt until the perforations open and allow fluid to move through.
Master Mix bead & mixing channel (shown in diagram 1510)—The freeze-dried master mix, which is an irregular sphere of ˜1 mm radius, is positioned at the end of the 2 mm column, just before the channel narrows to allow for easy assembly. Narrowing of the channel reduced the height-to-width ratio of the channel, speeding up the capillary-driven flow. The sample fluid is forced to dissolve a portion of the master mix as it moves through the channel. Pillars of 100 μm diameter enable mixing of the dissolved components to ensure a heterogeneous solution.
Sequential Trifurcation & mixing of dye & probes (shown in diagram 1512)—The fluid is then separated sequentially into 3 narrower subchannels, each of which contained solidified dye & probes set for varying targets of interest. Each dye and probe set is layered underneath the imaging chamber and dissolves in the sample solution upon contact. FIG. 26 illustrates a schematic of the trifurcation as well as results from a LAMP reaction performed using solidified dye & probe in a paper filter format.
Imaging chamber (shown in diagram 1514)—The imaging chamber consists of a serpentine channel or circular well geometry, through which the sample fluid (mixed with all the necessary constituents) moves through and is imaged. In the case of a serpentine channel, it can also be used to mix the sample fluid while providing a large enough space to hold>10 μl of reaction volume (20×3×0.2 mm) for imaging. The imaging chamber is detected using 4 corner markers.

The present inventors conducted fluid modelling and simulations to verify the workings of the microfluidic cartridge 110. Fluid flow was modeled and a time-dependent, two-phase, level set laminar flow regime was employed. Because the geometry of the microfluidic cartridge 110 is uniform in height (for example, 200 μm), a 2D model of the microfluidic cartridge 110 was rendered. The microfluidic cartridge 110 was separated into subcomponents for fluid flow modelling. For each subcomponent, a wetted wall condition was applied to all channel walls, except for the inlet and outlet. To ensure hydrophilicity, the contact angle of fluid on the inner surfaces of the device was selected to be 30 degrees: i.e., the empirically determined contact angle of water with a hydrophilic polymer. Polydimethylsiloxane, treated with O₂ plasma treatment prior to assembly. Gravity was included in the model at a value of −9.806 m/s². A pressure boundary condition was applied at the inlet. Initially, all channels were filled with air except for a portion of the channel following the inlet which was filled with fluid with properties of 0.9% NaCl solution. To model 0.9 % NaCl solution, a dynamic viscosity of 0.443 Pas and density of 0.981 g/cm³ were used. Finally, a temperature of 65° C. was used for all components.

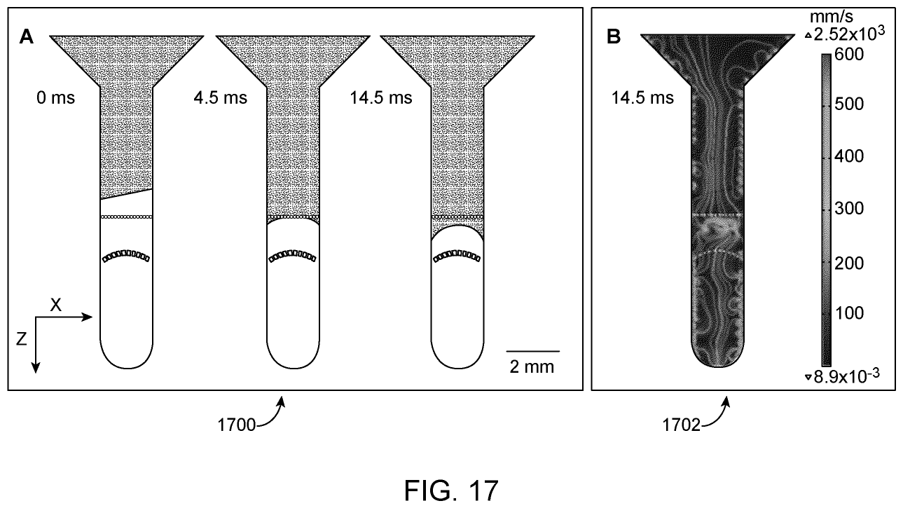

FIG. 17 illustrates a diagram of capillary-driven flow through the upper subcomponent of the microfluidic cartridge 110 at various time points to show movement of fluid from inlet through the bead filter, perforated plug and towards the narrowing mixing chamber. As shown in diagram 1700, sample fluid filling schematics show smooth filling with lack of bubbles as fluid passes through smaller pillar features. As shown in diagram 1702, velocity and streamline profile of fluid flow in mm/s, at 14.5 ms shows direction of fluid from inlet towards mixing chamber with limited obstructions. Model mesh details: 53.043 triangles: 1.340 edge elements: 121 vertex elements: 0.9404 average element quality.

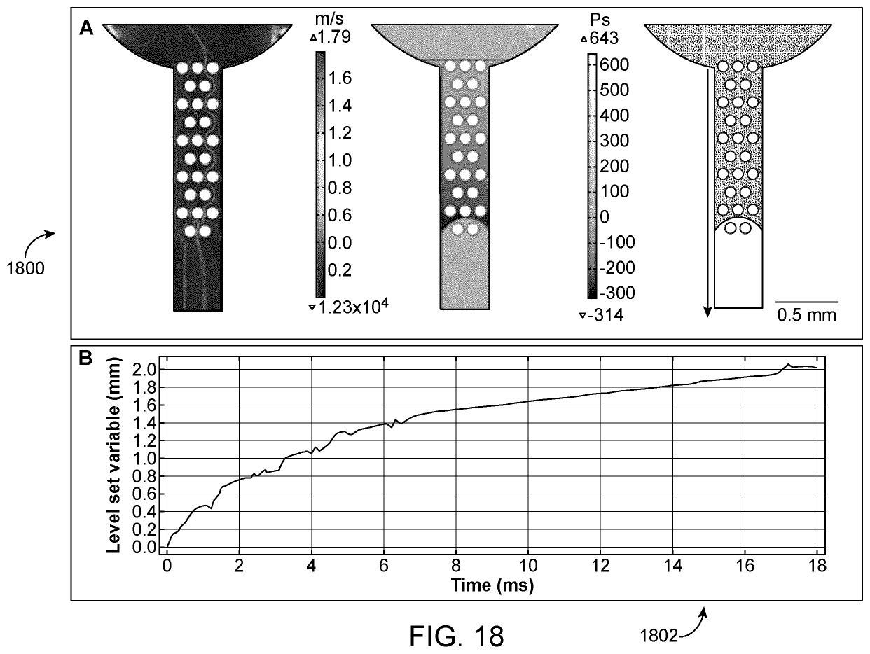

FIG. 18 illustrates a diagram of capillary -driven flow through the master mix bead mixing chamber. Diagram 1800 illustrates velocity profile and streamlines (left), pressure profile (centre) and volume fraction of fluid flow profile (right) of the mixing chamber demonstrate smooth movement of fluid with no bubbles. Diagram 1802 illustrates position of fluid meniscus over time, integrated along the left wall of the channel (indicated by arrow). Model mesh details: 14.967 triangles: 1.213 edge elements: 110 vertex elements: 0.8129 average element quality.

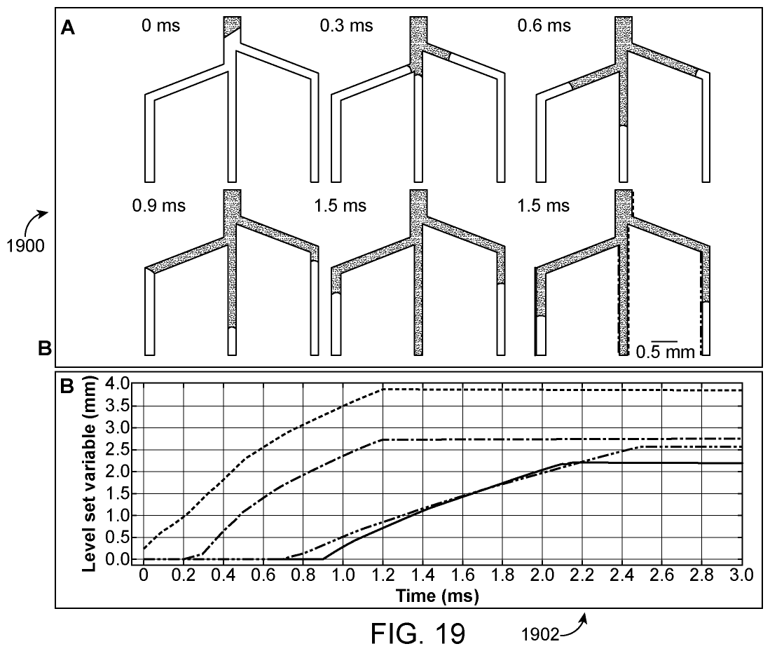

FIG. 19 illustrates capillary -driven flow through sequential trifurcation over time. Diagram 1900 illustrates volume fraction of fluid flow profile shows smooth filling of all subchannels with lack of bubbles. Diagram 1902 illustrates the position of fluid meniscus over time, integrated along the walls of the channel. Model mesh details; 10.004 triangles; 986 edge elements; 18 vertex elements; 0.924 average element quality.

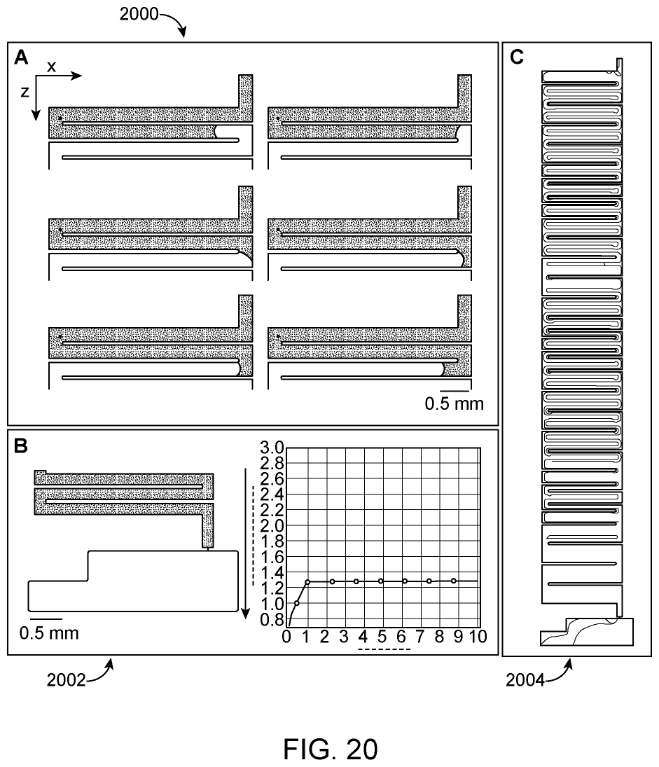

FIG. 20 illustrates capillary-driven flow through serpentine imaging chambers over time. Diagram 2000 illustrates volume fraction of fluid flow profile shows smooth filling of serpentine channel with preservation of contact angle along channel geometry. Diagram 2002 illustrates volume fraction of fluid flow profile (left) and position of fluid meniscus over time, integrated along the right-most walls of the channels (indicated by arrow). As fluid approaches the stop valve, the abrupt geometry changes prevent further capillary-driven flow. Instead, the position of the meniscus plateaus with time and remains constant thereafter. Diagram 2004 illustrates a streamline profile of fluid flow along imaging chambers. Model mesh details: 70.096 triangles; 11.059 edge elements; 337 vertex elements; 0.8767 average element quality.

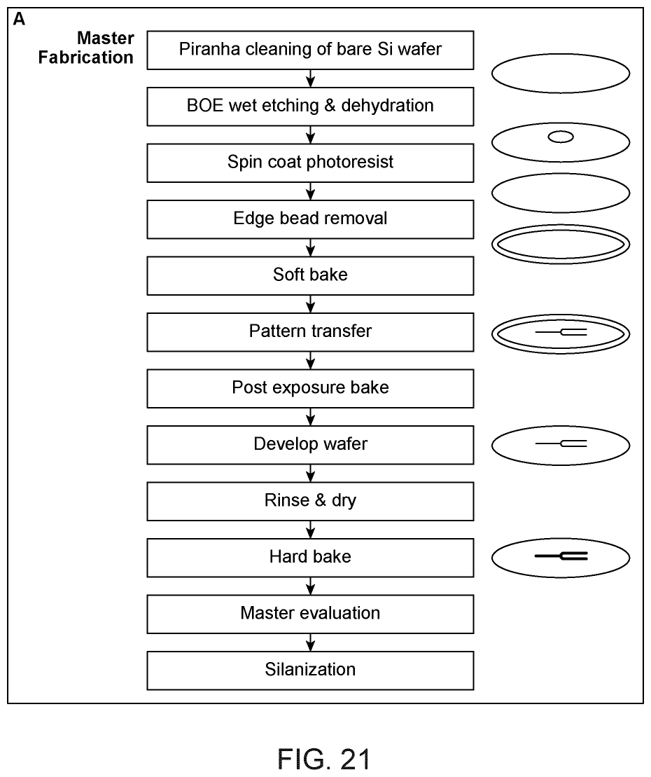

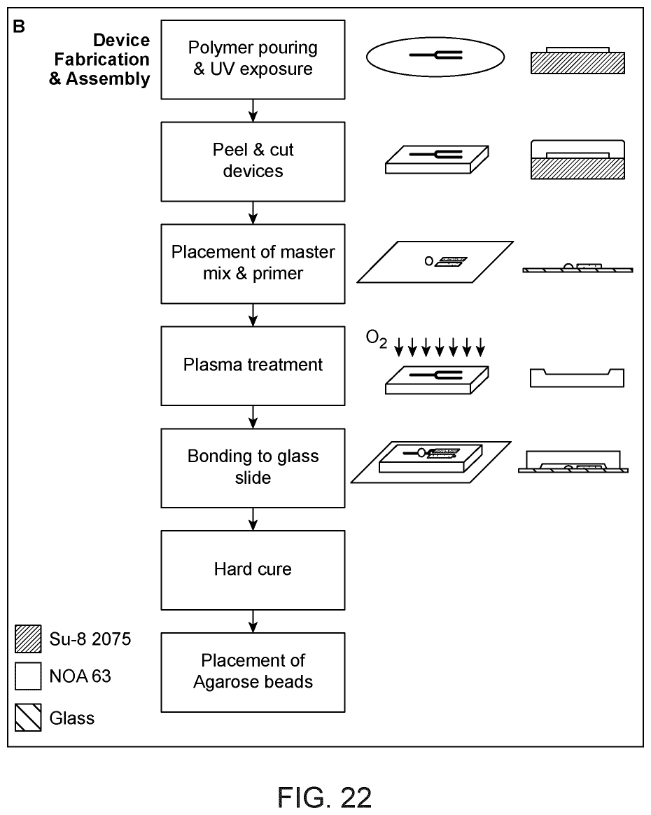

FIGS. 21 and 22 illustrate an example of a process flow and stepwise illustration for creating an SU-8 master (FIG. 21) and a microfluidic cartridge (FIG. 22) by photolithography and cast molding techniques. Fabrication and device assembly steps are outlined. The microfluidic cartridge is a hybrid of glass and NOA 63, a hydrophilic alternative to PDMS that gives a better contact angle in combination with plasma treatment. To fabricate the master, a 76.2 mm bare silicon wafer is cleaned with a 3:1 piranha solution of H₂SO₄: H₂O₂ (10 minutes), and a 10:1 buffer oxide etching solution (30 seconds). The silicon wafer is then dehydrated at 200° C. for 10 minutes. SU-8 2075 (3 ml) is dispensed unto the silicon wafer and spun at (1) 500 rpm for 5-10 seconds [100 rpm/s] and (2) 1250 rpm for 30 seconds [300 rpm/s]. The wafer is then baked at 65° C. for 5 minutes and 95° C. for 16 minutes. The pattern is transferred with UV exposure, and the wafer is baked again at 65° C. for 3 minutes 48 seconds and 95° C. for 9 minutes 12 seconds. The wafer is immersed in SU-8 developer for 8 minutes 48 seconds, rinsed with IPA and developer (10 seconds each) and hard baked at 150° C. for 30 minutes. The completed master is evaluated for quality under a microscope and profilometer. Prior to device fabrication, the master is silanized using trichlorofluorosilane (Cl3Fsi). NOA 63 is poured over the master and UV cured for 140 seconds. The polymer is peeled and cut to size. The polymer and a glass slide are plasma treated for 60 seconds at a pressure of 0.1 Torr with a plasma power of 20 W. The master mix bead and probe+dye are then correctly positioned over the glass slide. The polymer is bonded to the glass, allowing the probe+dye mix to take the shape of serpentine channels above it. The entire device is then UV cured for 2 hours.

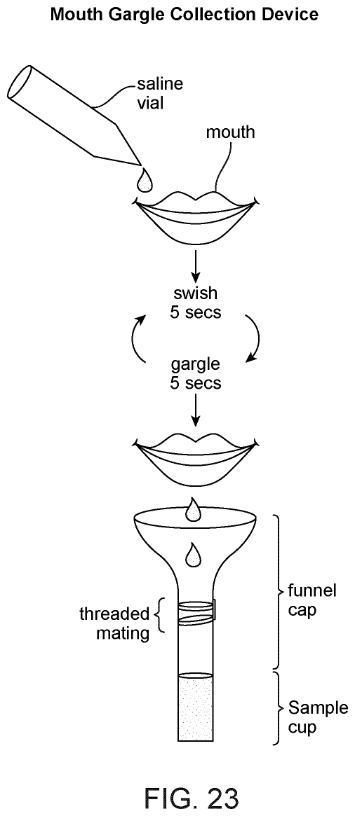

The present embodiments provide the collection device 108 that can use samples from a mouth rinse or a nasal rinse to ensure seamlessness and accuracy, with easy sample collection. FIG. 23 illustrates an embodiment of a mouth rinse collection device comprising a funnel or funnel cap and a sample cup, with a threaded mating connecting the two. Alternatively, the funnel and sample cup can be press fit together. The sample collection device can be used to obtain a test sample from a patient and to transfer that sample to a microfluidic cartridge. In accordance with this embodiment, the sample collection funnel is attached to the sample collection cup, a patient sample (e.g., a bodily fluid) is dispensed into the funnel, the funnel removed and the collection cup attached to the microfluidic cup. Subsequently, the patient sample can then be dispensed into the microfluidic cartridge, for example by using capillary action.

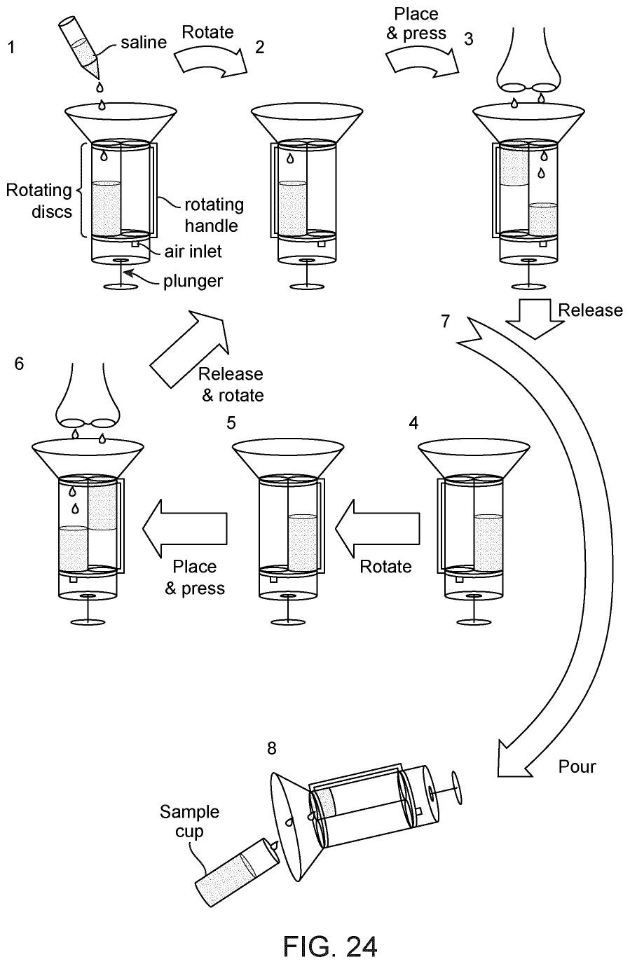

FIG. 24 illustrates a nasal rinse collection device comprising a funnel cap and two chambers, a reagent chamber and a sample collection chamber (or a sample collection cup). A plunger is located on the chambers opposite the funnel cap. Each of the chambers has an associated cap and is selected by a rotating handle. Upon depressing the plunger, the uncapped chamber has air forced in from an air inlet causing liquid therein to be ejected. The chambers are filled with saline solution, then the handle is rotated to place the nozzle inside the nasal cavity. Upon pressing on the plunger, pressurized saline solution is ejected into the nasal cavity. Solution travels the spaces that a nasopharyngeal swab would go through, cleans and dispenses the contents into the cup again. The process is repeated in the other nose hole to ensure samples from all the nasal space is collected. The sample collection cup can then be attached to a microfluid cartridge and the collected patient fluid sample transferred to the microfluidic cartridge, for example by using capillary action.

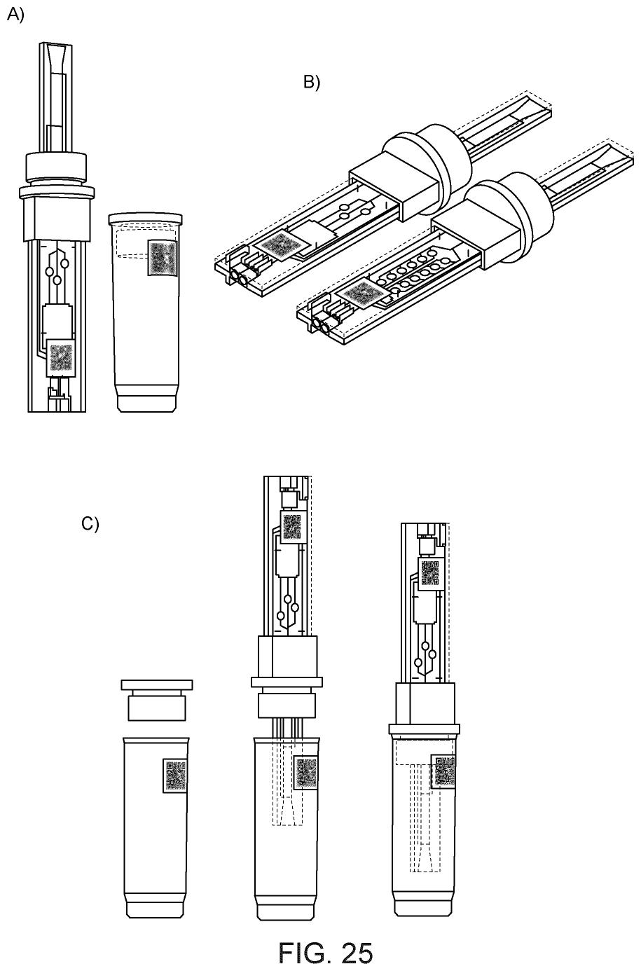

FIG. 25 illustrates an embodiment of a sample collection device comprising a sample collection cartridge and a sample cup containing a sample reagent and a computer-readable QR code side by side, an embodiment of the cartridge containing up to 12 circular imaging chambers and assembly of the cartridge and sample cup with one motion. As shown in FIG. 25, the sample collection device comprising a collection cartridge having one or more capillary channels for collecting a patient sample (e.g., a blood sample) and a sample cup. The sample collection device can be used to obtain a test sample from a patient and to transfer that sample to a microfluidic cartridge. In accordance with this embodiment, a patient's finger can be pricked to draw blood which is then brough into contact with an edge of the collection cartridge and a blood sample collected via capillary action. The sample collection cartridge is then inserted the sample cup containing the sample collection reagent and the reagent drawn from the sample cup into the capillary channels of the sample cartridge (e.g., using capillary action). The sample collection cup can then be attached to a microfluid cartridge and the collected patient fluid sample transferred to the microfluidic cartridge, for example by using capillary action.

Advantageously, FIG. 26 demonstrates collection of a small amount of liquid sample such as blood from finger prick by touching and gliding over the cartridge edge.

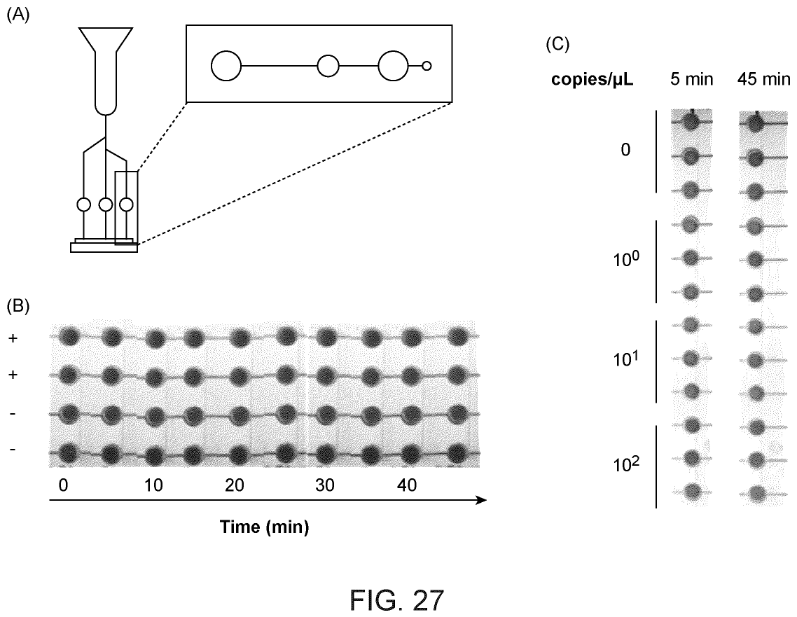

FIG. 27 illustrates a time-course RT-LAMP reaction and data collected from circular imaging chambers in order to detect SARS-COV-2 on paper matrices pre-treated with SARS-COV-2 primers and Eriochrome Black T dye. This embodiment detected 10 copies per microliter within 45 minutes.

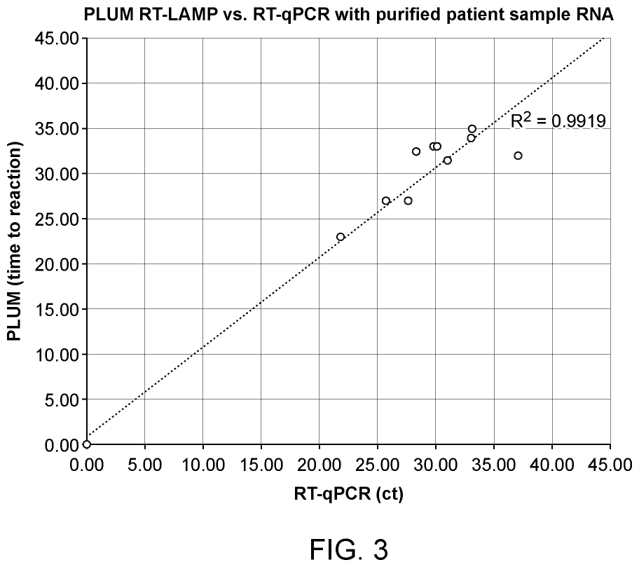

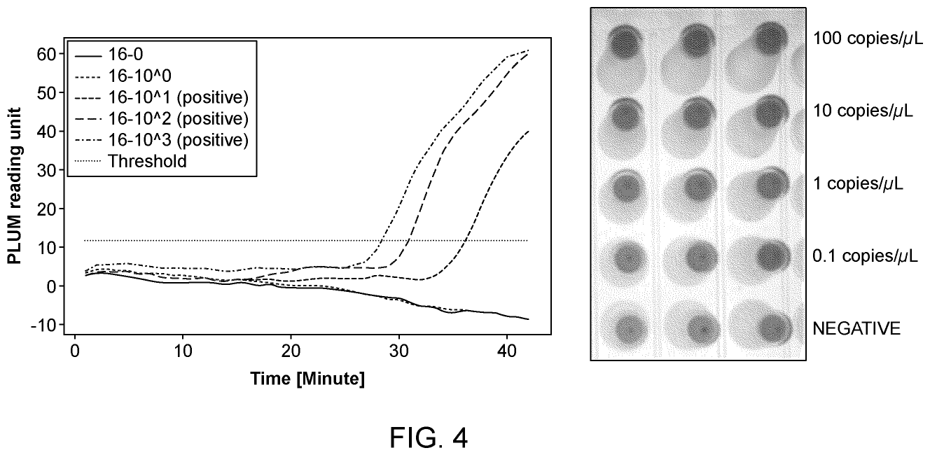

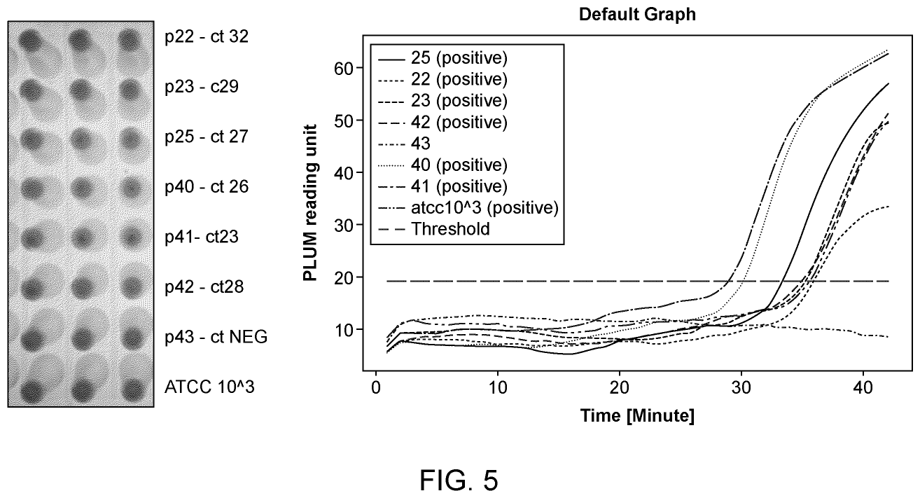

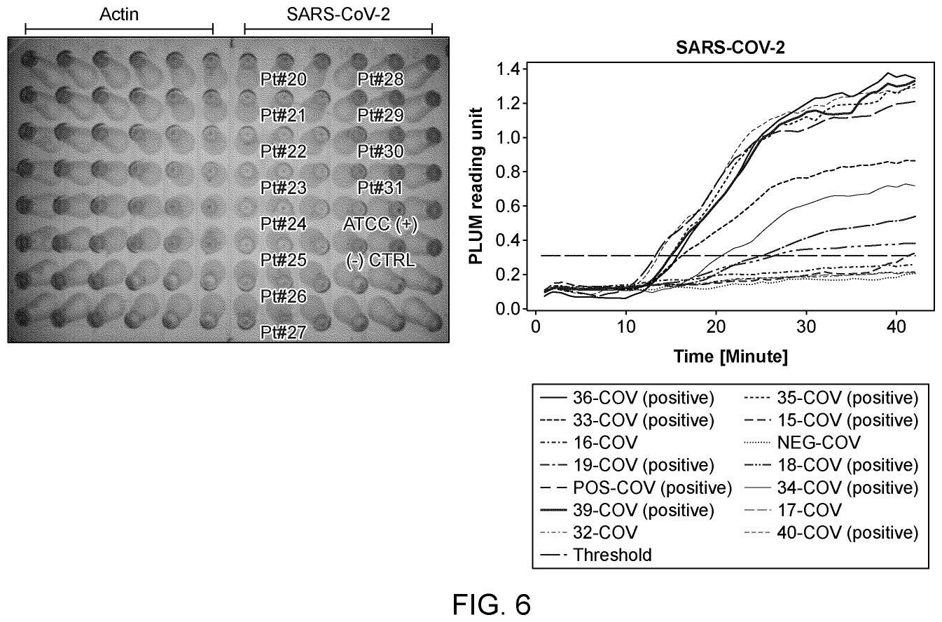

The present embodiments can advantageously use an RT-LAMP mixture which is compatible with freeze-drying. In an embodiment, it includes NEB M 1710B Custom WarmStart™ LAMP 4× Master Mix (Lyo-Compatible) in addition to D-(+)-Trehalose (Sigma Cat#T0167) and PEG 20000 (Sigma Cat#96172), EDTA, Tris (ph 8.0), and primers and Eriochrome Black T. FIG. 3 illustrates a correlation between RT-LAMP using the above freeze-dried formulation with RT-qPCR for the detection of SARS-COV-2 from patient samples (positive and negative) (n=14 samples). FIG. 4 illustrates a picture of samples and a chart of an example sensitivity test of the RT-LAMP performed in an analysis device using the above freeze-dried formulation. Amplification is seen using the above formulation down to ˜1 copy of virus/μL with a total reaction volume of 16 μL (ATCC, MP-32 SARS-COV-2 control). FIG. 5 illustrates a picture of samples and a chart of an example performance of RT-LAMP using the above freeze-dried formulation for the detection of SARS-COV-2 from gargle and swish patient samples. The samples are heat inactivated in the presence of TCEP (2.5 mM), EDTA (1 mM). The detection of SARS-COV-2 up to ct value 32 (validated using RT-qPCR). The image indicates positive amplification of the viral RNA and negative amplifications. ATCC. MP-32 SARS-CoV-2 were used as a positive control. FIG. 6 illustrates a picture of samples and a chart of an example from patient sample testing using RT-LAMP in an analysis device. The gargle and swish samples of 12 patients were tested (using B-Actin as an internal control for the experiment) for the presence of SARS-COV-2 in the sample. RT-LAMP was carried out using pH indicator based NEB colorimetric RT-LAMP mix with LAMP primers targeting E1 and ORF1A genes. A patient study was performed with N=246 with 65 validated using RT-qPCR.

The present inventors determined a series of experiments to determine an optimal formulation of RT-LAMP reactions for freeze drying. For freeze drying, NEB's glycerol free 4× RT-LAMP mix, was used as a base, and additives were included, such as PEG and trehalose. The experiments started off by comparing NEB's 4× glycerol free RT-LAMP mix (which is amenable to freeze-drying) with the 2×fluorescent and colorimetric RT-LAMP mixes (which cannot be freeze-dried). The primers were tested for B-Actin using extracted HEK 293 RNA, as well as extracted patient RNA with a corresponding Ct Value of 28.08. From this, it was determined that the 4× mix performed similarly to the 2× mixes.

| 2x Fluorescent | MM | 4x Fluorescent | MM | 2x Colorimetric | MM |
|---|---|---|---|---|---|
| Sample | Cq | Sample | Cq | Sample | Cq |
| 2x Fluoro HEK | 14.11 | 4x Fluoro HEK | 10.16 | 2x Color HEK | 10.09 |
| 2x Fluoro HEK | 14.00 | 4x Fluoro HEK | 10.23 | 2x Color HEK | 10.10 |
| 2x Fluoro #77 | 21.04 | 4x Fluoro #77 | 15.64 | 2x Color #77 | 15.00 |
| 2x Fluoro #77 | 21.59 | 4x Fluoro #77 | 15.49 | 2x Color #77 | 15.01 |
| 2x Fluoro neg | — | 4x Fluoro neg | — | 2x Color neg | — |
| 2x Fluoro neg | — | 4x Fluoro neg | — | 2x Color neg | — |

The example experiments tested the addition of 5% w/v PEG 20000, and when that was determined not to have an adverse effect on the performance of RT-LAMP, the addition of 10% w/v trehalose was added, which also did not adversely affect the TTR of the RT-LAMP reactions.

| | |
|---|---|
| With 5% v/v PEG | CTL PEG− |

| Sample | Cq | Sample | Cq |
|---|---|---|---|
| Hek PEG | 12.96 | HEK | 13.18 |
| Hek PEG | 13.05 | HEK | 13.15 |
| #93 PEG | 17.06 | #93 | 17.35 |
| #93 PEG | 18.17 | #93 | 17.19 |
| Neg PEG | 35.84 | Neg | 46.12 |
| Neg PEG | | Neg | 58.68 |

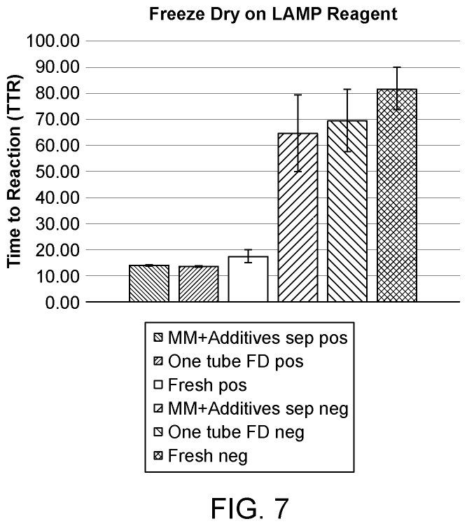

The example experiments then determined if the freeze-dried combination of both PEG and trehalose behaved similarly to the fresh enzyme mix, which we determined to be the case. A similar experiment was performed with Black T dye and the 4× MM, with the results illustrated in the chart of FIG. 7, and determined that the freeze dried and fresh experiments worked comparably.

The example experiments also tested rehydration of the freeze-dried (FD) samples using extracted RNA instead of H2O as the residual volume, comparing with FD and fresh controls. This was to see if there are any inhibitory effects with the addition of more RNA. The example experiments determined that there were ˜1 min inhibitory effects with low Ct value samples (24, 41) and high concentration viral RNA spike-ins (concentration 10\hat{ }3), with potentially some benefit in terms of being able to detect samples with high Ct values (26, 38).

20210304 FD Rehydration & Patient Sample RNA Test Fluoresce

| Freeze Dried, RNA | | | | | |
|---|---|---|---|---|---|
| Rehydration | | Fresh CTL | | qPCR1 | qPCR2 |
| Sample | Cq | Sample | Cq | Ct | Ct |
| 24 FD AVG | 14.60 | 24 AVG | 13.99 | 25.9 | 23.12 |
| 41 FD AVG | 13.20 | 41 AVG | 12.48 | 23.3 | 21.57 |
| 26 FD AVG | 18.75* | 26 AVG | 19.83 | 31.9 | 30.35 |
| 38 FD AVG | 18.13 | 38 AVG | — | 35.9 | — |
| 690 FD | — | 690 AVG | — | | |
| AVG | | | | | |
| H20 CTL | | H20 CTL | | | |
| FD | | | | | |

| Freeze Dried − RNA | | Freeze Dried | | | |
|---|---|---|---|---|---|
| Rehydration | | Control | | Fresh Control | |
| Sample | Cq | Sample | Cq | Sample | Cq |
| FD − RNA + | 24.67 | FD − 10\hat{ }3 | 13.97 | ATCC | 17.80 |
| 10\hat{ }3 | | | | 10\hat{ }3 | |
| FD − RNA + | 22.04 | FD − 10\hat{ }3 | 19.07 | ATCC | 16.82 |
| 10\hat{ }3 | | | | 10\hat{ }3 | |
| FD − RNA + | 22.24 | FD − 10\hat{ }3 | 17.13 | ATCC | 16.04 |
| 10\hat{ }3 | | | | 10\hat{ }3 | |
| FD − RNA + | 21.63 | FD − 10\hat{ }3 | 27.62 | ATCC | 17.78 |
| 10\hat{ }3 | | | | 10\hat{ }3 | |
| FD − RNA + | | FD − H20 | | H20 CTL | |
| CTL | | CTL | | | |
| FD − RNA + | | FD − H20 | | H20 CTL | |
| CTL | | CTL | | | |
| FD − RNA + | 43.31 | FD − H20 | | H20 CTL | |
| CTL | | CTL | | | |
| FD − RNA + | | FD − H20 | | H20 CTL | |
| CTL | | CTL | | | |

In order to freeze dry the sample, in an example, NEB 4× RT-LAMP MM is freeze dried with D-(+)-Trehalose (Sigma Cat#T0167) and PEG 20000 (Sigma Cat#96172) at a final concentration of 0.5% w/v of PEG and 10% w/v trehalose. More additives, including Tris and EDTA, can be freeze dried for magnesium-based dye indicators in RT-LAMP. The freeze drying can include placing the sample cup in −60C to −80C degree chamber and vacuum condition until all the water molecules are removed from the mixture.

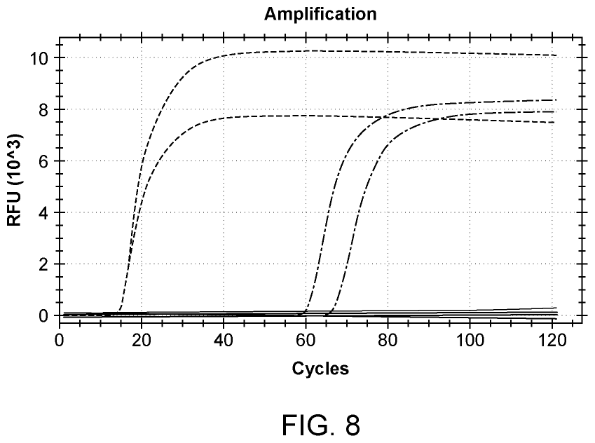

The example experiments also tested different hydrogels (Pluronic-F127, Gelatin, Agarose) for their ability to serve as a matrix for immobilizing the RT-LAMP primers and dye (either a fluorescence dye such as SYTO9 or Black-T). To this end, each hydrogel was first tested for its ability to conform to the temperature parameters of the RT-LAMP experiment, meaning that the hydrogel had to solidify and be in a gel at room temperature (˜20-24C) yet be liquid at 65C. Pluronic F-127, a poloxamer, failed these parameters, as its gelling properties fell outside of these requirements since a concentration could not be found at which it was solid at room temperature and liquid at 65C. It is possible that combining Pluronic F-127 with another hydrogel might yield a hydrogel with the desired properties. Hydrogels were also tested for any inhibitory effects to the RT-LAMP. Gelatin, at 5% and 7% w/w in water displayed appropriation thermal gelation properties, however inhibited the fluorescent RT-LAMP assay at both concentrations after being spiked into the final reaction as illustrated in the chart of FIG. 8 and in the following table:

12/02/21 RT-LAMP 5% 7.5% Gelatin Test

| CTL | | 5% gelatin | | 7.5% gelatin | |
|---|---|---|---|---|---|
| Sample | Cq | Sample | Cq | Sample | Cq |
| ATCC | 14.03 | ATCC 10\hat{ }3 + 5% | 5.45 (?) | ATCC 10\hat{ }3 + 7.5% | — |
| 10\hat{ }3 | | | | | |
| ATCC | 13.84 | ATCC 10\hat{ }3 + 5% | 116.64 | ATCC 10\hat{ }3 + 7.5% | — |
| 10\hat{ }3 | | | | | |
| H20 CTL | 65.05 | H20 CTL + 5% | 117.51 | H20 CTL + 7.5% | 118.24 |
| H20 CTL | 58.89 | H20 CTL + 5% | 115.19 | H20 CTL + 7.5% | — |

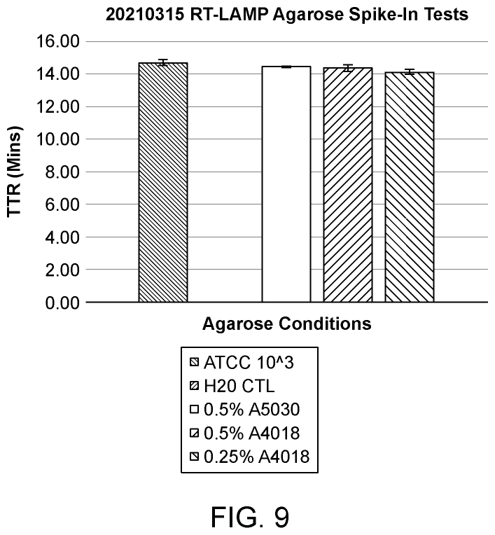

The example experiments also tested low melting temperature agarose, and displayed the thermal gelling properties at concentrations 0.25%- 0.5%. No inhibitory effects to the fluorescent RT-LAMP reaction (utilising SYTO9 dye) were seen after spiking in small amounts of the different low melting agarose types (A5030 and A4018 ), as illustrated in FIG. 9.

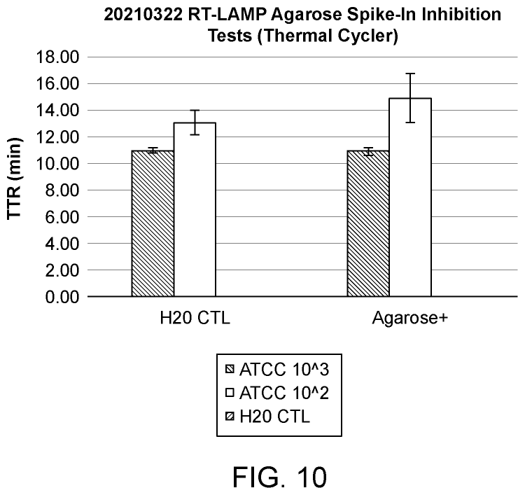

The example experiments then tested the addition of the entire primer/dye combination (in liquid form) to the enzyme mix (4×NEB MM. PEG, and Trehalose-fresh, not freeze dried) to see if there were any inhibitory effects. It was determined that there does not appear to be a significant decrease to the TTR (time to reaction) with the addition of agarose at a final concentration of 0.11% per reaction. however, there might be a reduction in overall fluorescence. As illustrated in FIG. 10. these experiments were also tested in an analysis device. where at lower concentrations the overall fluorescence intensity was reduced. without any impact to the TTR.

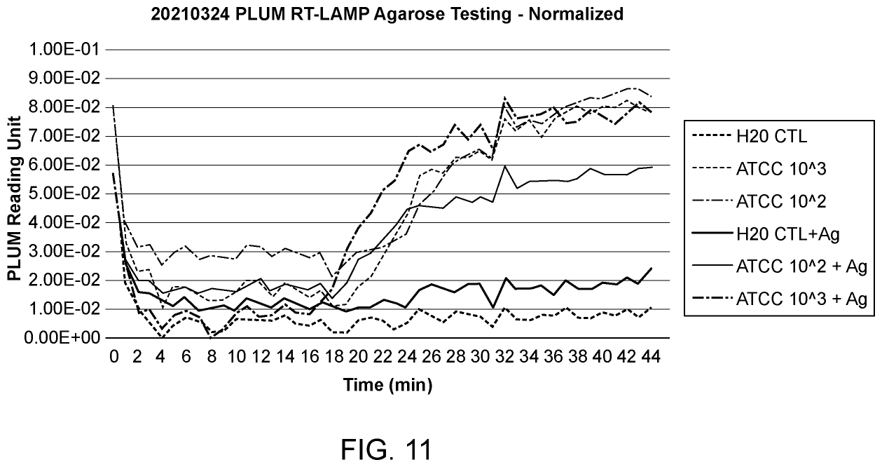

The example experiments then tested if having the agarose solidify prior to the addition of the enzyme mix would have an effect on the TTR or sensitivity. The primer/dye mix was formulated with a final agarose% of 0.25% and allowed to solidify in a 96 well plate prior to the addition of viral RNA. and then the enzyme mix. As illustrated in FIG. 11. it was observed that at higher concentrations of virus RNA. pre-gelled primers/dye appeared to result in a faster TTR. however at lower viral copies. there was no difference between the solidified and liquid primer/dye mix. indicating that prior dye/gel mix solidification doesn't negatively impact the TTR of RT-LAMP reactions.

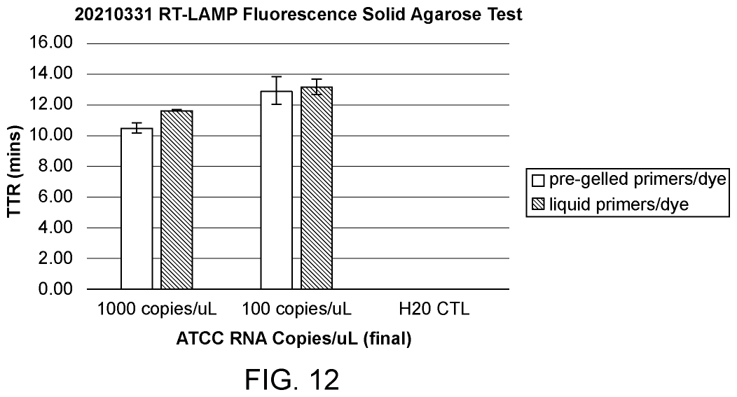

The example experiments then tested the sensitivity of the combination of the solidified primer/SYTO9 dye mix and the freeze-dried enzyme mix. Following success with increasing the RT-LAMP sensitivity by increasing the overall reaction volumes from 10 μl to 16 μl. the reaction volumes were increased for all subsequent experiments involving agarose testing. while still maintaining the final agarose% in the primer/dye mix (0.25%) and in the final reaction (0.11%). As before, the primer/dye mix was allowed to solidify in the wells of a 96 well plate. prior to the addition of 1.6 μL of RNA at final concentrations of 100 copy/μL to 0. copy/μL). As illustrated in FIG. 12. it was determined that it could reliably detect down to 1 copy/μL of SARS-COV-2 RNA (16 copies total).

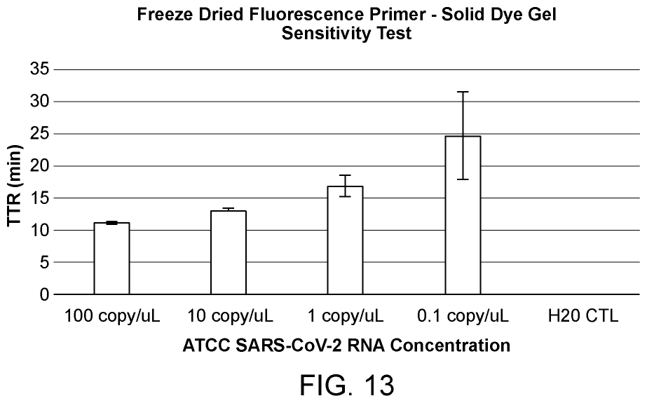

The example experiments then attempted to determine the sensitivity of the combination of the solidified primer/Black T dye mix and the freeze-dried enzyme mix. As illustrated in FIG. 13. it was observed that the initial phase separation of the primer/dye and the enzyme mix delays the initial magnesium concentration from being adjusted to purple. meaning that instead of undergoing a single-color shift from purple to blue. the reactions were starting from blue. turning purple. and returning to blue again. This might explain the lower sensitivity (ATCC 10 3-1600 SARS COV-2 copies) that was observed in comparison to experiments with the SYTO9 fluorescent dye. Since the final formulation in the microfluidic cartridge will generally not include phase separation between the primer/dye mix and enzyme mix. it is likely that this will not be an issue.

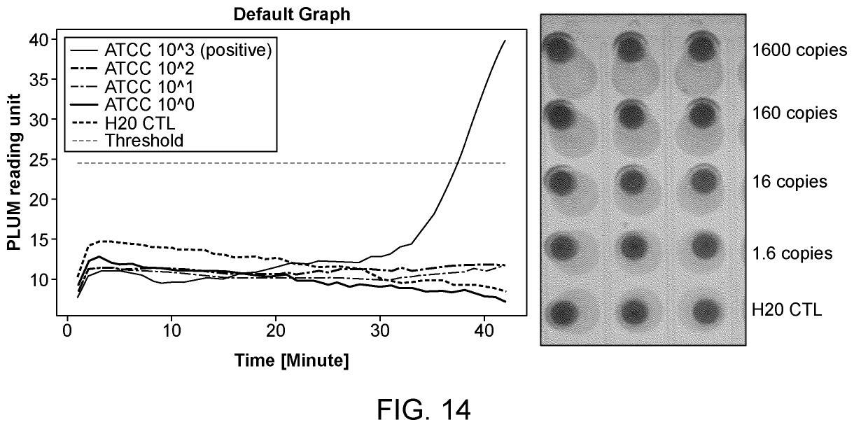

The example experiments then determined a protocol for agarose formulation, as illustrated in FIG. 14. RT-LAMP Primers specific for the diagnostic target, dye (5 μM final concentration for SYTO9 dye and 120 μM for Black-T). and low-melting temperature agarose (Sigma-Aldrich A914) were combined to a final volume of 0.25% w/v agarose in 5 μL. The agarose was prepared at a stock concentration of 0.49% w/v in nuclease-free water, briefly boiled at 100C to dissolve the agarose. and kept at a temperature >37 C to prevent solidification of the gel prior to addition to the primers and the dye. After combination. the primer-dye matrix was allowed to solidify at room temperature (˜20-25C). Upon heating to a temperature of 65C. the primer-dye matrix re-liquefies allowing for microfluidic manipulation and combination with the rehydrated RT-LAMP master mix and sample and for the reaction to proceed.

The present embodiments can advantageously use a BSA-treated paper matrix as an environment to solidify primers and dye combinations. This is achieved by spotting prepared formulation onto the pre-cut paper discs and drying the assembly as demonstrated in FIG. 27.

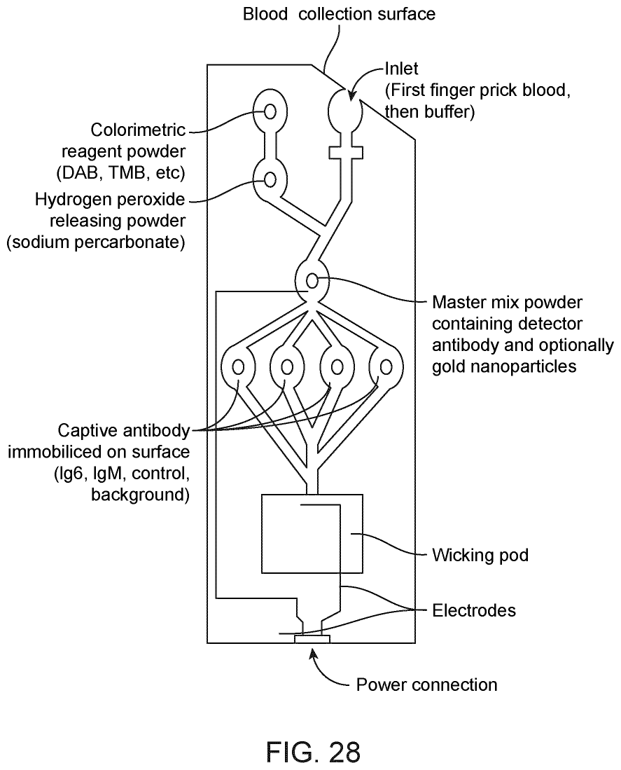

Alternatively, the master mix can include detector antibodies to capture disease specific antibodies while probes can be replaced with capture antigens or antibodies to allow detection of target biomarkers, i.e. antibodies such as COVID-19 IgG and IgM. FIG. 28 illustrates an embodiment of antibody detection that includes colorimetric detection reagent (such as 3,3′-diaminobenzidine or 3,3′, 5,5′-Tetramethylbenzidine) and hydrogen peroxide generating powders to facilitate colorimetric output generation. Advantageously, the embodiment also includes electrodes capable of receiving voltage differential to enable charge based reversible motion of analytes up and down the cartridge, enabling sweeping or traditional shaking motion.

Outputs of the cartridge can be detected by using enzymatic colorimetric reactions, binding of fluorescent molecules, bioluminescent agents or sequestering of gold nanoparticles.

Although the foregoing has been described with reference to certain specific embodiments, various modifications thereto will be apparent to those skilled in the art without departing from the spirit and scope of the invention as outlined in the appended claims. The entire disclosures of all references recited above are incorporated herein by reference.

1. A method for molecular diagnostics, the method comprising:
receiving a patient sample in a collection device;
coupling the collection device to a microfluidic cartridge;
dispensing the patient sample into the microfluidic cartridge using capillary action;
inserting the microfluidic cartridge into a diagnostic system;
detecting the microfluidic cartridge and initiating a sample testing protocol, wherein initiating the sample testing protocol comprises;
reading a computer readable code located on the microfluidic cartridge;
receiving a sample test protocol from a computer system based on the
computer readable code; and
performing the sample test protocol;
identifying a detection chamber or imaging chamber on the microfluidics device;
capturing image data of the patient sample in the detection chamber or imaging chamber at one or more time points during an incubation period based on the sample test protocol;
performing image analysis on the captured image data; and
outputting a diagnostic result based on the image analysis.

2. The method of claim 1, wherein the microfluidic cartridge comprises a sensor having a sensor surface, and optionally wherein the sensor surface comprises a cartridge surface, an immobilization surface or a porous paper matrix.

3. The method of claim 2, wherein the sensor surface comprises an immobilization surface, and wherein the immobilization surface contains agarose, gelatin, alginate, optical fiber, plastic surface or paper matrices.

4. The method of claim 1, wherein the detection chamber or imaging chamber comprises one or more regions of interest.

5. The method of claim 1, wherein the microfluidic cartridge contains pillars, a porous matrix or membrane for separation by size exclusion of particles larger than viral particles.

6. The method of claim 1, wherein the sample testing protocol comprises one or more test parameters selected from assay conditions, assay temperature, incubation time, image capture parameters, illumination sources, optical filters or any combination thereof.

7. The method of claim 6, wherein the image capture parameters comprise fluorescent, luminescent or colorimetric, and wherein the captured image data is fluorescent data, colorimetric data, wavelength data, bioluminescent data or chemiluminescent data.

8. The method of claim 1, wherein the sample testing protocol comprises the use of one or more reagents and wherein the one or more reagents are added to the patient sample to create a reaction sample.

9. The method of claim 8, wherein the one or more reagents comprises probes or primers, and wherein the probes or primers are stored in a liquid medium or immobilized to the sensor surface.

10. The method of claim 8, wherein the one or more reagents comprises capture antibodies, and wherein the capture antibodies are stored in a liquid medium or immobilized to the sensor surface.

11. The method of claim 8, wherein the one or more reagents comprises a freeze-dried master mix comprising antibodies, enzymes or gold nanoparticles, and wherein the antibodies, enzymes or gold nanoparticles facilitate a change in the captured image data.

12. The method of claim 11, wherein the freeze-dried master mix is mixed with the patient sample by passing the patient sample through a microfluidic channel in the microfluidic cartridge containing the freeze-dried master mix thereby creating a reaction sample by mixing of the patient sample with the freeze-dried master mix.

13. The method of claim 8, wherein the one or more reagents comprises a colorimetric reagent and/or a hydrogen peroxide reagent, and wherein the colorimetric reagent and/or hydrogen peroxide reagent are stored on the microfluidic cartridge in one or more reagent storage compartments.

14. The method of claim 1, wherein the testing protocol further comprises inactivating the sample in the microfluidic cartridge, and wherein the inactivation comprises chemical, physical or thermal inactivation.

15. The method of claim 14, wherein chemical inactivation comprises incubation with a detergent and/or chelating agent.

16. The method of claim 14, wherein physical inactivation comprises sonication.

17. The method of claim 1, wherein the testing protocol further comprises performing multiplexed detection of different targets by passing the sample into different detection chambers, optionally wherein each of said detection chambers comprises a different reagent.

18. The method of claim 1, wherein the computer system comprises a processor, storage and computer readable code, and wherein the computer readable code includes one or more sample testing protocols.

19. The method of claim 1. wherein the sample collection device further comprises a computer readable code, and wherein the sample collection device computer readable code is read and linked to a patient sample or patient reference number to facilitate tracking of the patient sample and wherein optionally the computer readable code is used as a token to communicate with an external database.

20. The method of claim 1, wherein the microfluidic cartridge computer readable code is further linked to test quality information including but not limited to a cartridge serial number to facilitate automate processing of the patient sample.

21. The method of claim 1, wherein the diagnostic results are stored in the cloud, and optionally wherein the diagnostic results further comprise an associated reference number, one or more quality information features, such as batch number, expiry date, lot number, or successful analysis threshold, and one or more operator information features, such as operator ID, and optionally wherein the test results are matched with an external database for patient identification.

22. The method of claim 2, wherein the microfluidic cartridge comprises two wire leads capable of receiving a voltage differential in order to move charged particles inside the device reversibly.

23. The method of claim 2, wherein the microfluidic cartridge comprises two wire leads capable of receiving a voltage differential in order to heat, lyse particles, or for flow control by manipulating temperature or voltage sensitive materials.

24. The method of claim 2, wherein the diagnostic device contains electrical connectors that mate with leads of an external device to detect the presence of the microfluidic cartridge.

25.-55. (canceled)
